(19) United States
(12) Patent Application Publication
(10) Pub. No.: US 2008/0009479 A1
(43) Pub. Date: Jan. 10, 2008

(54) Tetrahydrobenzazepines as Histamine H3 Receptor Ligands

(75) Inventors: Thomas Daniel Heightman, Essex (GB); David Matthew Wilson, Essex (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(73) Assignee: GLAXO GROUP LIMITED, Greenford, Middlesex (GB)

(21) Appl. No.: 10/599,636

(22) PCT Filed: Apr. 6, 2005
(86) PCT No.: PCT/GB05/01333

§ 371 Date: Oct. 4, 2006

(30) Foreign Application Priority Data
Apr. 8, 2004 (GB) 0408083.4

(51) Int. Cl. A61K 31/55; A61P 25/00; C07D 223/16

(52) U.S. Cl. 514/217.01; 540/594

(57) ABSTRACT

The present invention relates to novel benzazepine derivatives having pharmacological activity, processes for their preparation, to compositions containing them and to their use in the treatment of neurological and psychiatric disorders.

The present invention relates to novel benzazepine derivatives having pharmacological activity, processes for their preparation, to compositions containing them and to their use in the treatment of neurological and psychiatric disorders.

JP 2001226269 and WO 00/23437 (Takeda Chem Ind Ltd) describe a series of benzazepine derivatives which are claimed to be useful in the treatment of obesity. DE 2207430, U.S. Pat. No. 4,210,749 and FR 2171879 (Pennwalt Corp) and GB 1268243 (Wallace and Tiernan Inc) all describe a series of benzazepine derivatives which are claimed as being antagonists for narcotics (such as morphine or codeine) and also anti-histamines and anticholinergic agents. WO 02/14513 (Takeda Chem Ind Ltd) describe a series of benzazepine derivatives with GPR12 activity which are claimed to be useful in the treatment of attention deficit disorder, narcolepsy or anxiety. WO 02/02530 (Takeda Chem Ind Ltd) describe a series of benzazepine derivatives as GPR14 antagonists which are claimed to be useful in the treatment of hypertension, atherosclerosis and cardiac infarction. WO 01/03680 (Isis Innovation Ltd) describe a series of benzazepine derivatives which are claimed as effective agents in the preparation of cells for transplantation in addition to the inhibition of diseases such as diabetes. WO 00/21951 (SmithKline Beecham plc) discloses a series of tetrahydrobenzazepine derivatives as modulators of dopamine D3 receptors which are claimed to be useful as antipsychotic agents. WO 01/87834 (Takeda Chem Ind Ltd) describe a series of benzazepine derivatives as MCH antagonists which are claimed to be useful in the treatment of obesity. WO 02/15934 (Takeda Chem Ind Ltd) describe a series of benzazepine derivatives as urotensin II receptor antagonists which are claimed to be useful in the treatment of neurodegenerative disorders. WO 04/018432 (Eli Lilly and Company) describe a series of substituted azepines as histamine H3 receptor antagonists for the treatment of obesity and other histamine H3 receptor related diseases. WO 03/090751 (Pfizer Products Inc.) describe a series of N-substituted heteroaryloxy-aryloxy-pyrimidine-2,4,6-trione metalloproteinase inhibitors and their use for the treatment of inflammation and cancer. WO 2004/026305 (Eli Lilly and Company) describe a series of diaryl ethers as opioid receptor antagonists and their use for the treatment of obesity.

The histamine H3 receptor is predominantly expressed in the mammalian central nervous system (CNS), with minimal expression in peripheral tissues except on some sympathetic nerves (Leurs et al., (1998), Trends Pharmacol. Sci. 19, 177-183). Activation of H3 receptors by selective agonists or histamine results in the inhibition of neurotransmitter release from a variety of different nerve populations, including histaminergic and cholinergic neurons (Schlicker et al., (1994), Fundam. Clin. Pharmacol. 8, 128-137). Additionally, in vitro and in vivo studies have shown that H3 antagonists can facilitate neurotransmitter release in brain areas such as the cerebral cortex and hippocampus, relevant to cognition (Onodera et al., (1998), In: The Histamine H3 receptor, ed Leurs and Timmerman, pp 255-267, Elsevier Science B.V.). Moreover, a number of reports in the literature have demonstrated the cognitive enhancing properties of H3 antagonists (e.g. thioperamide, clobenpropit, ciproxifan and GT-2331) in rodent models including the five choice task, object recognition, elevated plus maze, acquisition of novel task and passive avoidance (Giovanni et al., (1999), Behav. Brain Res. 104, 147-155). These data suggest that novel H3 antagonists and/or inverse agonists such as the current series could be useful for the treatment of cognitive impairments in neurological diseases such as Alzheimer's disease and related neurodegenerative disorders.

The present invention provides, in a first aspect, a compound of formula (i) or a pharmaceutically acceptable salt thereof:

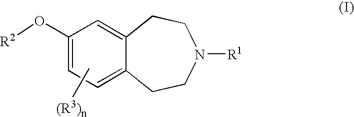

wherein:

R¹ represents —C₂₋₇ alkyl or —(CH₂)ₘ—C₃₋₇ cycloalkyl;

R² represents —X—C₃₋₈ cycloalkyl, —X-aryl, —X-heteroaryl, —X-heterocyclyl, —X—C₃₋₈ cycloalkyl-Y—C₃₋₈ cycloalkyl, —X—C₃₋₈ cycloalkyl-Y-aryl, —X—C₃₋₈ cycloalkyl-Y-heteroaryl, —X—C₃₋₈ cycloalkyl-Y-heterocyclyl, —X-aryl-Y—C₃₋₈ cycloalkyl, —X-aryl-Y-aryl, —X-aryl-Y-heteroaryl, —X-aryl-Y-heterocyclyl, —X-heteroaryl-Y—C₃₋₈ cycloalkyl, —X-heteroaryl-Y-aryl, —X-heteroaryl-Y-heteroaryl, —X-heteroaryl-Y-heterocyclyl, —X-heterocyclyl-Z-aryl, —X-heterocyclyl-Y—C₃₋₈ cycloalkyl, —X-heterocyclyl-Y-heteroaryl or —X-heterocyclyl-W-heterocyclyl, such that R² is linked to O via a carbon atom;

W represents a bond, C₁₋₆ alkyl, CO, COC₂₋₆ alkenyl, O or SO₂;

X represents a bond or C₁₋₆ alkyl;

Y represents a bond, C₁₋₆ alkyl, CO, COC₂₋₆ alkenyl, O or SO₂;

Z represents a bond, CO, COC₂₋₆ alkenyl, O or SO₂;

R³ represents halogen, C₁₋₆ alkyl, C₁₋₆ alkoxy, cyano, amino or trifluoromethyl;

m represents an integer from 1-3;

n is 0, 1 or 2;

wherein said alkyl groups of R¹ may be optionally substituted by one or more substituents (eg. 1, 2 or 3) which may be the same or different and which are selected from the group consisting of halogen, cyano, ═O, C₁₋₆ alkyl, C₁₋₆ alkoxy, haloC₁₋₆ alkyl or haloC₁₋₆ alkoxy;

wherein said cycloalkyl, aryl, heteroaryl and heterocyclyl groups of R² may be optionally substituted by one or more substituents (eg. 1, 2 or 3) which may be the same or different, and which are selected from the group consisting of halogen, hydroxy, cyano, nitro, ═O, trifluoromethyl, trifluoromethoxy, fluoromethoxy, difluoromethoxy, C₁₋₆ alkyl, pentafluoroethyl, C₁₋₆ alkoxy, arylC₁₋₆ alkoxy, C₁₋₆ alkylthio, C₁₋₆ alkoxyC₁₋₆ alkyl, C₃₋₇ cycloalkylC₁₋₆ alkoxy, C₁₋₆ alkanoyl, C₁₋₆ alkoxycarbonyl, C₁₋₆ alkylsulfonyl, C₁₋₆ alkylsulfinyl, C₁₋₆ alkylsulfonyloxy, C₁₋₆ alkylsulfonylC₁₋₆ alkyl, sulfonyl, arylsulfonyl, arylsulfonyloxy, arylsulfonylC₁₋₆ alkyl, aryloxy, C₁₋₆ alkylsulfonamido, C₁₋₆ alkylamino, C₁₋₆ alkylamido, —R⁴, —CO₂R⁴, —COR⁴, C₁₋₆ alkylsulfonamidoC₁₋₆ alkyl, C₁₋₆ alkylamidoC₁₋₆ alkyl, arylsulfonamido, arylcarboxamido, arylsulfonamidoC₁₋₆ alkyl, arylcarboxamidoC₁₋₆ alkyl, aroyl, aroylC₁₋₆ alkyl, arylC₁₋₆ alkanoyl, or a group —NR⁵R⁶, —C₁₋₆ alkyl-NR⁵R⁶, —C₃₋₈ cycloalkyl-NR⁵R⁶, —NR⁵COR⁶, —NR⁵SO₂R⁶, —OCONR⁵R⁶, —NR⁵CO₂R⁶, —NR⁴CONR⁵R⁶ or —SO₂NR⁵R⁶ (wherein R⁴, R⁵ and R⁶ independently represent hydrogen, C₁₋₆ alkyl, —C₃₋₈ cycloalkyl, —C₁₋₆ alkyl-C₃₋₈ cycloalkyl, aryl, heterocyclyl or heteroaryl or wherein-NR⁵R⁶ may represent a nitrogen containing heterocyclyl group, wherein said R⁴, R⁵ and R⁶ groups may be optionally substituted by one or more substituents (eg. 1, 2 or 3) which may be the same or different, and which are selected from the group consisting of halogen, hydroxy, C₁₋₆ alkyl, C₁₋₆ alkoxy, cyano, amino, ═O or trifluoromethyl);

or solvates thereof.

In one aspect:

R¹ represents —C₂₋₇ alkyl or —(CH₂)ₘ—C₃₋₇ cycloalkyl;

R² represents —X—C₃₋₈ cycloalkyl, —X-aryl, —X-heteroaryl, —X—C₃₋₈ cycloalkyl-Y—C₃₋₈ cycloalkyl, —X—C₃₋₈ cycloalkyl-Y-aryl, —X—C₃₋₈ cycloalkyl-Y-heteroaryl, —X—C₃₋₈ cycloalkyl-Y-heterocyclyl, —X-aryl-Y—C₃₋₈ cycloalkyl, —X-aryl-Y-aryl, —X-aryl-Y-heteroaryl, —X-aryl-Y-heterocyclyl, —X-heteroaryl-Y—C₃₋₈ cycloalkyl, —X-heteroaryl-Y-aryl, —X-heteroaryl-Y-heteroaryl, —X-heteroaryl-Y-heterocyclyl, —X-heterocyclyl-Z-aryl, —X-heterocyclyl-Y-heteroaryl or —X-heterocyclyl-W-heterocyclyl, such that R² is linked to O via a carbon atom;

W represents C₁₋₆ alkyl, CO, COC₂₋₆ alkenyl, O or SO₂;

X represents a bond or C₁₋₆ alkyl;

Y represents a bond, C₁₋₆ alkyl, CO, COC₂₋₆ alkenyl, O or SO₂;

Z represents a bond, CO, COC₂₋₆ alkenyl, O or SO₂;

R³ represents halogen, C₁₋₆ alkyl, C₁₋₆ alkoxy, cyano, amino or trifluoromethyl;

m represents an integer from 1-3;

n is 0, 1 or 2;

wherein said alkyl groups of R¹ may be optionally substituted by one or more substituents (eg. 1, 2 or 3) which may be the same or different and which are selected from the group consisting of halogen, cyano, ═O, C₁₋₆ alkyl, C₁₋₆ alkoxy, haloC₁₋₆ alkyl or haloC₁₋₆ alkoxy;

wherein said cycloalkyl, aryl, heteroaryl and heterocyclyl groups of R² may be optionally substituted by one or more substituents (eg. 1, 2 or 3) which may be the same or different, and which are selected from the group consisting of halogen, hydroxy, cyano, nitro, ═O, trifluoromethyl, trifluoromethoxy, fluoromethoxy, difluoromethoxy, C₁₋₆ alkyl, pentafluoroethyl, C₁₋₆ alkoxy, arylC₁₋₆ alkoxy, C₁₋₆ alkylthio, C₁₋₆ alkoxyC₁₋₆ alkyl, C₃₋₇ cycloalkylC₁₋₆ alkoxy, C₁₋₆ alkanoyl, C₁₋₆ alkoxycarbonyl, C₁₋₆ alkylsulfonyl, C₁₋₆ alkylsulfinyl, C₁₋₆ alkylsulfonyloxy, C₁₋₆ alkylsulfonylC₁₋₆ alkyl, sulfonyl, arylsulfonyl, arylsulfonyloxy, arylsulfonylC₁₋₆ alkyl, aryloxy, C₁₋₆ alkylsulfonamido, C₁₋₆ alkylamino, C₁₋₆ alkylamido, —R⁴, —CO₂R⁴, —COR⁴, C₁₋₆ alkylsulfonamidoC₁₋₆ alkyl, C₁₋₆ alkylamidoC₁₋₆ alkyl, arylsulfonamido, arylcarboxamido, arylsulfonamidoC₁₋₆ alkyl, arylcarboxamidoC₁₋₆ alkyl, aroyl, aroylC₁₋₆ alkyl, arylC₁₋₆ alkanoyl, or a group —NR⁵R⁶, —C₁₋₆ alkyl-NR⁵R⁶, —C₃₋₈ cycloalkyl-NR⁵R⁶, —CONR⁵R⁶, —NR⁵COR⁶, —NR⁵SO₂R⁶, —OCONR⁵R⁶, —NR⁵CO₂R⁶, —NR⁴CONR⁵R⁶ or —SO₂NR⁵R⁶ (wherein R⁴, R⁵ and R⁶ independently represent hydrogen, C₁₋₆ alkyl, —C₃₋₈ cycloalkyl, —C₁₋₆ alkyl-C₃₋₈ cycloalkyl, aryl, heterocyclyl or heteroaryl or wherein-NR⁵R⁶ may represent a nitrogen containing heterocyclyl group, wherein said R⁴, R⁵ and R⁶ groups may be optionally substituted by one or more substituents (eg. 1, 2 or 3) which may be the same or different, and which are selected from the group consisting of halogen, hydroxy, C₁₋₆ alkyl, C₁₋₆ alkoxy, cyano, amino, ═O or trifluoromethyl).

In one embodiment, where R² is X-aryl-Y-heterocyclyl or X-heteroaryl-Y-heterocyclyl (wherein X is a bond and Y is O, CO, CH₂ or SO₂), and in one embodiment where R² is X-aryl or X-heteroaryl (wherein X is a bond and the aryl or heteroaryl group is substituted by COR⁴, wherein R⁴ is a heterocyclyl group), the heterocyclyl group is not pyrimidine-2,4,6-trione or a substituted pyrimidine-2,4,6-trione.

In one embodiment in which R² is X-aryl or X-heteroaryl (wherein X is a bond and the heteroaryl group is a nitrogen containing heteroaryl group), the aryl group or nitrogen containing heteroaryl group is not substituted by —COR⁴ (wherein R⁴ is a nitrogen containing heterocyclyl group) or a —CONR⁵R⁶ group.

In a further aspect, the cycloalkyl, aryl, heteroaryl and heterocyclyl groups of R² may be optionally substituted by one or more substituents (eg. 1, 2 or 3) which may be the same or different, and which are selected from the group consisting of halogen, hydroxy, cyano, nitro, ═O, C₁₋₆ alkoxy, phenylC₁₋₆ alkoxy, C₁₋₆ alkylthio, C₁₋₆ alkoxyC₁₋₆ alkyl, C₃₋₇ cycloalkylC₁₋₆ alkoxy, C₁₋₆ alkylsulfonyl, C₁₋₆ alkylsulfinyl, C₁₋₆ alkylsulfonyloxy, C₁₋₆ alkylsulfonylC₁₋₆ alkyl, sulfonyl, phenylsulfonyl, phenylsulfonyloxy, phenylsulfonylC₁₋₆ alkyl, phenyloxy, —R⁴, —CO₂R⁴, —COR⁴, C₁₋₆ alkylsulfonamidoC₁₋₆ alkyl, C₁₋₆ alkylamidoC₁₋₆ alkyl, phenylsulfonamidoC₁₋₆ alkyl, phenylcarboxamidoC₁₋₆ alkyl, phenylcarbonylC₁₋₆ alkyl, phenylC₁₋₆ alkanoyl, or a group —NR⁵R⁶, —C₁₋₆ alkyl-NR⁵R⁶, —C₃₋₈ cycloalkyl-NR⁵R⁶, —CONR⁵R⁶, —NR⁵COR⁶, —NR⁵SO₂R⁶, —OCONR⁵R⁶, —NR⁵CO₂R⁶, —NR⁴CONR⁵R⁶ or —SO₂NR⁵R⁶ (wherein R⁴, R⁵ and R⁶ independently represent hydrogen, C₁₋₆ alkyl, 13 C₃₋₈ cycloalkyl, —C₁₋₆ alkyl-C₃₋₈ cycloalkyl, aryl, heterocyclyl or heteroaryl and wherein said R⁴, R⁵ and R⁶ groups may be optionally substituted by one or more substituents (eg. 1, 2 or 3) which may be the same or different, and which are selected from the group consisting of halogen, hydroxy, C₁₋₆ alkyl, C₁₋₆ alkoxy, cyano, amino, ═O or trifluoromethyl);

Alkyl groups, whether alone or as part of another group, may be straight chain or branched and the groups alkoxy and alkanoyl shall be interpreted similarly. Alkyl moieties are more preferably C₁₋₄ alkyl, eg. methyl or ethyl. The term ‘halogen’ is used herein to describe, unless otherwise stated, a group selected from fluorine, chlorine, bromine or iodine.

References to ‘aryl’ include references to monocyclic carbocyclic aromatic rings (eg. phenyl) and bicyclic carbocyclic aromatic rings (e.g. naphthyl) or carbocyclic benzofused rings (eg. C₃₋₈ cycloalkyl fused to a phenyl ring, such as dihydroindenyl or tetrahydronaphthalenyl).

The term “heterocyclyl” is intended to mean a 4-7 membered monocyclic saturated or partially unsaturated aliphatic ring or a 4-7 membered saturated or partially unsaturated aliphatic ring fused to a benzene ring, which aliphatic ring contains 1 to 3 heteroatoms selected from oxygen, nitrogen or sulphur. Suitable examples of such monocyclic rings include pyrrolidinyl, piperidinyl, piperazinyl, morpholinyl, thiomorpholinyl, tetrahydrofuranyl, tetrahydropyranyl, diazepanyl, azepanyl, imidazolidinyl, isothiazolidinyl, oxazolidinyl, pyrrolidinone and tetrahydro-oxazepinyl. Suitable examples of benzofused heterocyclic rings include indolinyl, isoindolinyl, benzodioxolyl, dihydroisoindole, dihydrobenzofuranyl, dihydrobenzothiopyranyl and dihydroisoquinolinyl.

The term “nitrogen containing heterocyclyl” is intended to represent any heterocyclyl group as defined above which contains a nitrogen atom.

The term “heteroaryl” is intended to mean a 5-7 membered monocyclic aromatic or a fused 8-11 membered bicyclic aromatic ring, which monocyclic or bicyclic aromatic ring contains 1 to 3 heteroatoms selected from oxygen, nitrogen and sulphur. Suitable examples of such monocyclic aromatic rings include thienyl, furyl, pyrrolyl, triazolyl, imidazolyl, oxazolyl, thiazolyl, oxadiazolyl, isothiazolyl, isoxazolyl, thiadiazolyl, pyrazolyl, pyrimidyl, pyridazinyl, pyrazinyl, pyridyl and tetrahydropyranyl. Suitable examples of such fused aromatic rings include benzofused aromatic rings such as quinolinyl, isoquinolinyl, quinazolinyl, quinoxalinyl, cinnolinyl, naphthyridinyl, indolyl, indazolyl, furopyridinyl, pyrrolopyridinyl, benzofuranyl, benzothienyl, benzimidazolyl, benzoxazolyl, benzisoxazolyl, benzothiazolyl, benzisothiazolyl, benzoxadiazolyl, benzothiadiazolyl and the like.

In one aspect, R¹ represents:
—(CH₂)ₘ—C₃₋₇ cycloalkyl (eg. —CH₂-cyclopropyl or —CH₂-cyclobutyl); or
—C₂₋₇ alkyl (eg. 1-methyl ethyl, 2-methyl propyl, ethyl, 2,2-dimethyl propyl or 1-methyl propyl)

In a further aspect, m represents 1.

In one embodiment, R² represents —X-aryl, —X-aryl-Y-heterocyclyl, —X-heteroaryl, —X-heteroaryl-Y-heterocyclyl, —X-heterocyclyl, —X-heterocyclyl-Z-aryl, —X-heteroaryl-Y-heteroaryl, —X-heterocyclyl-W-heterocyclyl, —X-heterocyclyl-Y—C₃₋₈ cycloalkyl or —X—C₃₋₈ cycloalkyl.

X may represent a bond or —C₁₋₆ alkyl (eg, methyl or propyl), most typically a bond.

Y may represent a bond, CO, SO₂ or —CO—CH═CH—. In a more particular aspect, Y represents a bond or CO, and in a most particular aspect, Y represents a bond.

Z typically represents CO, and W typically represents CO.

R² may be unsubstituted or substituted with one or more substitutents selected from the group consisting of halogen, ═O, cyano, C₁₋₆alkyl, C₁₋₆ alkoxycarbonyl or a —CONR⁵R⁶ group.

In a more particular embodiment, R² represents:
—X-aryl (eg. —CH₂-phenyl or -phenyl) optionally substituted by halogen (eg. iodine) or alkoxycarbonyl (eg. ethoxycarbonyl);
—X-aryl-Y-heterocyclyl (eg. phenyl-imidazolyl) optionally substituted by methyl or an ═O group;
—X-heteroaryl (eg. pyridinyl or pyrazinyl) optionally substituted by a —CONR⁵R⁶ group (eg. CON(H)Me);
—X-heteroaryl-Y-heterocyclyl (eg. -pyrazinyl-pyrrolidinyl or -pyridinyl-pyrrolidinyl) optionally substituted by an ═O group;
—X-heterocyclyl (eg. -piperidinyl or —CH₂— piperidinyl) optionally substituted by alkoxycarbonyl (eg. tert-butoxycarbonyl);
—X-heterocyclyl-Z-aryl (eg -piperidinyl-CO-phenyl or —CH₂-piperidinyl-CO-phenyl) optionally substituted by cyano or halogen (eg. fluorine)
—X-heteroaryl-Y-heteroaryl (eg. pyridinyl-oxadiazolyl) optionally substituted by methyl;
—X-heterocyclyl-W-heterocyclyl (eg. —CH₂-piperidinyl-CO-tetrahydropyranyl);
—X-heterocyclyl-Y—C₃₋₈ cycloalkyl (eg. —CH₂-piperidinyl-CO-cyclopropyl); or
—X—C₃₋₈ cycloalkyl (eg. propyl-cyclohexyl).

In a most particular embodiment, R² represents:
—X-aryl (eg. —CH₂-phenyl or -phenyl) optionally substituted by halogen (eg. iodine) or alkoxycarbonyl (eg. ethoxycarbonyl);
—X-aryl-Y-heterocyclyl (eg. phenyl-N-imidazolyl) optionally substituted with one or more substitutents selected from methyl and ═O (eg. -phenyl-N-(3-methyl imidazolidin-2-one));
—X-heteroaryl (eg. -3-pyridinyl or -2-pyrazinyl) optionally substituted by a —CONR⁵R⁶ group (eg. CON(H)Me);
—X-heteroaryl-Y-heterocyclyl (eg. -2-pyrazinyl-N-pyrrolidinyl, -2-pyridinyl-N-pyrrolidinyl or -3-pyridinyl-N-pyrrolidinyl) optionally by substituted by an ═O group (eg. -2-pyrazinyl-N-pyrrolidin-2-one, -2-pyridinyl-N-pyrrolidin-2-one or -3-pyridinyl-N-pyrrolidin-2-one);
—X-heterocyclyl (eg. -4-piperidinyl or —CH₂-4-piperidinyl) optionally substituted by alkoxycarbonyl (eg. tert-butoxycarbonyl);
—X-heterocyclyl-Z-aryl (eg -4-piperidinyl-CO-phenyl or —CH₂-4-piperidinyl-CO-phenyl) optionally substituted by cyano or halogen (eg. fluorine)
—X-heteroaryl-Y-heteroaryl (eg. -2-pyridinyl-1,2,4-oxadiazol-5-yl) optionally substituted by methyl;
—X-heterocyclyl-W-heterocyclyl (eg. —CH₂-4-piperidinyl-CO-tetrahydropyran-4-yl);
—X-heterocyclyl-Y—C₃₋₈ cycloalkyl (eg. —CH₂-4-piperidinyl-CO-cyclopropyl); or
—X—C₃₋₈ cycloalkyl (eg. propyl-cyclohexyl).

In one embodiment, n represents 0 or 1. In a more particular embodiment, n represents 0.

When n represents 1, R³ is typically a halogen (eg. iodine) atom or a cyano group.

In one aspect:

R¹ represents —CH₂—C₃₋₇ cycloalkyl or —C₂₋₇ alkyl;

R² represents —X-aryl, —X-aryl-Y-heterocyclyl, —X-heteroaryl, —X-heteroaryl-Y-heterocyclyl, —X-heterocyclyl, —X-heterocyclyl-Z-aryl, —X-heteroaryl-Y-heteroaryl, —X-heterocyclyl-W-heterocyclyl, —X-heterocyclyl-Y—C₃₋₈ cycloalkyl or —X—C₃₋₈ cycloalkyl;

X represents a bond;

Y represents a bond;

Z represents CO;

W represents CO;

n represents 0;

wherein R² may be unsubstituted or substituted with one or more substitutents selected from the group consisting of halogen, ═O, cyano, C₁₋₆alkyl, C₁₋₆ alkoxycarbonyl or a —CONR⁵R⁶group.

Compounds according to the invention include the compounds of examples E1-32 as shown below, or a pharmaceutically acceptable salt thereof.

Compounds according to the invention include:

1-(5-{[3-(Cyclopropylmethyl)-2,3,4,5-tetrahydro-1H-3-benzazepin-7-yl]oxy}-2-pyrazinyl)-2-pyrrolidinone;

1-(6-{[3-(Cyclopropylmethyl)-2,3,4,5-tetrahydro-1H-3-benzazepin-7-yl]oxy}-3-pyridinyl)-2-pyrrolidinone;

1-{6-[(3-Ethyl-2,3,4,5-tetrahydro-1H-3-benzazepin-7-yl)oxy]-3-pyridinyl}-2-pyrrolidinone; and

1-(6-{[3-(1-methylpropyl)-2,3,4,5-tetrahydro-1H-3-benzazepin-7-yl]oxy}-3-pyridinyl)-2-pyrrolidinone.

Compounds of formula (I) may form acid addition salts with acids, such as conventional pharmaceutically acceptable acids, for example maleic, hydrochloric, hydrobromic, phosphoric, acetic, fumaric, salicylic, sulphate, citric, lactic, mandelic, tartaric and methanesulphonic. Salts, solvates and hydrates of compounds of formula (I) therefore form an aspect of the invention.

Certain compounds of formula (I) are capable of existing in stereoisomeric forms. It will be understood that the invention encompasses all geometric and optical isomers of these compounds and the mixtures thereof including racemates. Tautomers also form an aspect of the invention.

The present invention also provides a process for the preparation of a compound of formula (I) or a pharmaceutically acceptable salt thereof, which process comprises:

(a) reacting a compound of formula (II)

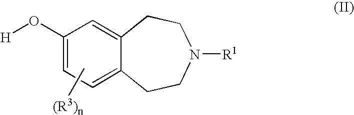

wherein R¹, R³ and n are as defined above, with a compound of formula R^{2′}-L¹, wherein R^{2′} is as defined above for R² or a group convertible thereto and L¹ represents a suitable leaving group such as a halogen atom (eg. bromine or iodine) or an optionally activated hydroxyl group;

(b) reacting a compound of formula (III)

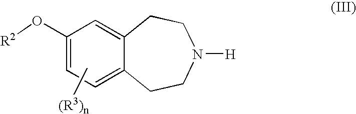

wherein R², R³ and n are as defined above, with a compound of formula R^{1′}-L², wherein R^{1′} is as defined above for R¹ or a group convertible thereto and L² represents a suitable leaving group such as a halogen atom (eg. bromine, iodine or tosylate); or

(c) reacting a compound of formula (III) as defined above, with a ketone of formula R^{1″}═O, wherein R^{1″} is ═C₂₋₇ alkyl or ═(CH₂)ₘ—C₃₋₇ cycloalkyl or a group convertible thereto; or

(d) deprotecting a compound of formula (I) which is protected; or

(e) interconversion from one compound of formula (I) to another.

When the leaving group L¹ is attached to an sp³ hybridised carbon, for example, R^{2′}-L¹ is an alkyl halide, process (a) typically comprises the use of a suitable base, such as potassium carbonate in an appropriate solvent such as 2-butanone optionally in the presence of a catalyst such as potassium iodide at an appropriate temperature such as reflux.

When the leaving group L¹ is attached to an sp² hybridised carbon, for example, R^{2′}-L¹ is an aryl halide, process (a) typically comprises the use of a copper(I) salt, such as copper (I) iodide, in the presence of a base such as sodium hydride, in an appropriate solvent such as pyridine, at an appropriate temperature such as reflux.

When the leaving group L¹ is attached to an activated sp² hybridised carbon for example, R^{2′}-L¹ is a heteroaryl halide such as a 2-chloropyridine or 2-chloropyrazine, process (a) typically comprises the use of a suitable base, such as sodium hydride in an appropriate solvent such as dimethylformamide or dimethyl sulfoxide, at an appropriate temperature. Alternatively, potassium tert-butoxide in tert-butanol at an appropriate temperature may also be employed.

When the leaving group L¹ is attached to an activated sp² hybridised carbon, for example R^{2′}-L¹ is an aryl halide such as 3,4-difluoro-benzonitrile, process (a) typically comprises the use of a suitable base, potassium carbonate, in a suitable solvent, such as dimethyl sulfoxide, at a suitable temperature.

When L¹ is a hydroxyl group attached to an sp³ hybridised carbon, for example, R^{2′}-L¹ is an alcohol, process (a) typically comprises the use of a phosphine such as triphenylphosphine in a suitable solvent such as tetrahydrofuran, followed by addition of an azodicarboxylate such as diethylazodicarboxylate, di-t-butylazodicarboxylate or bis (1,1-dimethylethyl) 1,2-diazenedicarboxylate at a suitable temperature such as room temperature.

Process (b) typically comprises the use of a suitable base, such as potassium carbonate in an appropriate solvent such as 2-butanone optionally in the presence of a catalyst such as potassium iodide at an appropriate temperature such as reflux.

Process (c) typically comprises the use of reductive conditions (such as treatment with a borohydride eg. sodium triacetoxyborohydride or (polystyrylmethyl)trimethylammonium cyanoborohydride), optionally in the presence of an acid, such as acetic acid, in an appropriate solvent such as dichloromethane at a suitable temperature such as room temperature.

In process (d), examples of protecting groups and the means for their removal can be found in T. W. Greene ‘Protective Groups in Organic Synthesis’ (J. Wiley and Sons, 1991). Suitable amine protecting groups include sulphonyl (e.g. tosyl), acyl (e.g. acetyl, 2′,2′,2′-trichloroethoxycarbonyl, benzyloxycarbonyl or t-butoxycarbonyl) and arylalkyl (e.g. benzyl), which may be removed by hydrolysis (e.g. using an acid such as hydrochloric acid in dioxan or trifluoroacetic acid in dichloromethane) or reductively (e.g. hydrogenolysis of a benzyl group or reductive removal of a 2′,2′,2′-trichloroethoxycarbonyl group using zinc in acetic acid) as appropriate. Other suitable amine protecting groups include trifluoroacetyl (—COCF₃) which may be removed by base catalysed hydrolysis or a solid phase resin bound benzyl group, such as a Merrifield resin bound 2,6-dimethoxybenzyl group (Ellman linker), which may be removed by acid catalysed hydrolysis, for example with trifluoroacetic acid.

Process (e) may be performed using conventional interconversion procedures such as epimerisation, oxidation, reduction, alkylation, nucleophilic or electrophilic aromatic substitution, ester hydrolysis, amide bond formation or transition metal mediated coupling reactions. Examples of transition metal mediated coupling reactions useful as interconversion procedures include the following: Palladium catalysed coupling reactions between organic electrophiles, such as aryl halides, and organometallic reagents, for example boronic acids (Suzuki cross-coupling reactions); Palladium catalysed amination and amidation reactions between organic electrophiles, such as aryl halides, and nucleophiles, such as amines and amides; Copper catalysed amidation reactions between organic electrophiles (such as aryl halides) and nucleophiles such as amides; and Copper mediated coupling reactions between phenols and boronic acids.

Compounds of formula (II) and (III) may be prepared in accordance with the following scheme

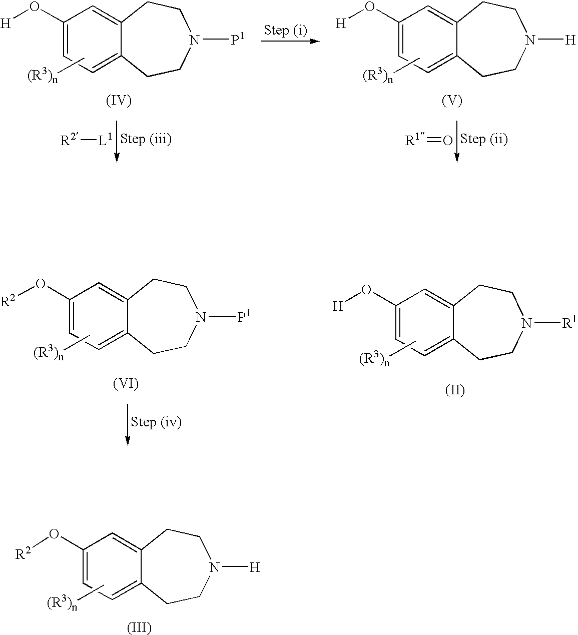

wherein R′, R¹″, R², R^{2′}, R³, n and L¹ are as defined above and P¹ represents a suitable protecting group such as Boc.

Step (i) typically comprises a deprotection reaction. Examples of protecting groups and means for their removal were described in relation to process (d). When P¹ represents Boc the deprotection reaction may comprise reaction of a compound of formula (IV) with an acid, for example hydrochloric acid in dioxan or trifluoroacetic acid in dichloromethane.

Step (ii) may be performed under reducing conditions in an analogous manner to that described for process (c).

Step (iii) may be performed in an analogous manner to that described for process (a).

Step (iv) typically comprises a deprotection reaction to provide a compound of formula (III) and can be performed as described in step (i).

Compounds of formula (VI) wherein R² represents —X-aryl, —X-heteroaryl, —X-aryl-Y—C₃₋₈ cycloalkyl, —X-aryl-Y-aryl, —X-aryl-Y-heteroaryl, —X-aryl-Y-heterocyclyl, —X-heteroaryl-Y—C₃₋₈ cycloalkyl, —X-heteroaryl-Y-aryl, —X-heteroaryl-Y-heteroaryl or —X-heteroaryl-Y-heterocyclyl and X represents a bond may also be prepared in accordance with the following scheme

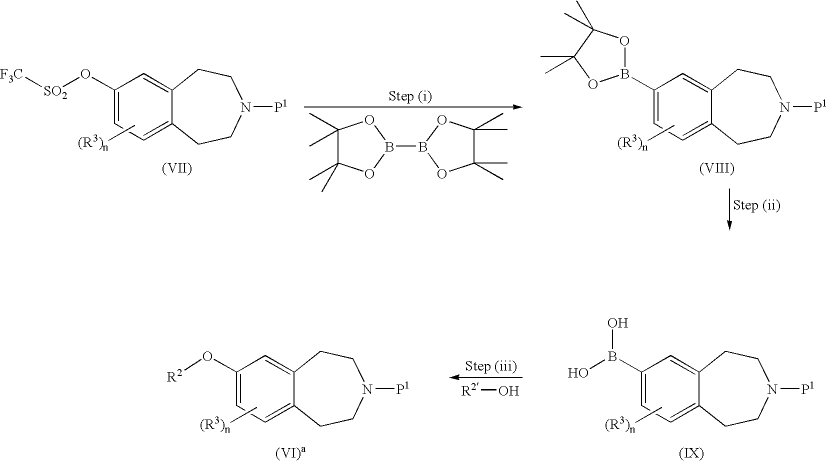

wherein R², R^{2′}, R³ and n are as defined above and P¹ represents a suitable protecting group such as Boc.

Step (i) may be performed under palladium catalysed cross-coupling conditions, for example using bis(diphenylphosphino)ferrocenedichloropalladium (II) complex and 1,1′-bis(diphenylphosphino)ferrocene as the catalyst system, in combination with a suitable base, such as potassium acetate, in a suitable solvent, for example dioxane, at a suitable temperature, for example reflux.

Step (ii) may be performed under oxidising conditions, for example using sodium periodate in the presence of ammonium acetate, in a suitable solvent system, such as acetone and water, at a suitable temperature, for example room temperature.

Step (iii) may be performed in the presence of a copper salt, for example copper acetate, in combination with a suitable base, such as triethylamine, together with molecular sieves, in a suitable solvent, for example dichloromethane, at a suitable temperature, for example room temperature.

Compounds of formula (IV) may be prepared in an analogous manner to those described in Description 3 of WO 02/40471.

Compounds of formula (VII) may be prepared as outlined in Bioorg. Med. Chem. Lett.; 10; 22; 2000; 2553-2556.

Compounds of formula (I) and their pharmaceutically acceptable salts have affinity for and are antagonists and/or inverse agonists of the histamine H3 receptor and are believed to be of potential use in the treatment of neurological diseases including Alzheimer's disease, dementia, age-related memory dysfunction, mild cognitive impairment, cognitive deficit, epilepsy, neuropathic pain, inflammatory pain, migraine, Parkinson's disease, multiple sclerosis, stroke and sleep disorders including narcolepsy; psychiatric disorders including schizophrenia (particularly cognitive deficit of schizophrenia), attention deficit hyperactivity disorder, depression and addiction; and other diseases including obesity, asthma, allergic rhinitis, nasal congestion, chronic obstructive pulmonary disease and gastrointestinal disorders.

Thus the invention also provides a compound of formula (I) or a pharmaceutically acceptable salt thereof, for use as a therapeutic substance in the treatment or prophylaxis of the above disorders, in particular cognitive impairments in diseases such as Alzheimer's disease and related neurodegenerative disorders.

The invention further provides a method of treatment or prophylaxis of the above disorders, in mammals including humans, which comprises administering to the sufferer a therapeutically effective amount of a compound of formula (I) or a pharmaceutically acceptable salt thereof.

In another aspect, the invention provides the use of a compound of formula (I) or a pharmaceutically acceptable salt thereof in the manufacture of a medicament for use in the treatment of the above disorders.

When used in therapy, the compounds of formula (I) are usually formulated in a standard pharmaceutical composition. Such compositions can be prepared using standard procedures.

Thus, the present invention further provides a pharmaceutical composition for use in the treatment of the above disorders which comprises the compound of formula (I) or a pharmaceutically acceptable salt thereof and a pharmaceutically acceptable carrier.

The present invention further provides a pharmaceutical composition which comprises the compound of formula (I) or a pharmaceutically acceptable salt thereof and a pharmaceutically acceptable carrier.

Compounds of formula (I) may be used in combination with other therapeutic agents, for example histamine H1 antagonists or medicaments claimed to be useful as either disease modifying or symptomatic treatments of Alzheimer's disease. Suitable examples of such other therapeutic agents may be agents known to modify cholinergic transmission such as 5-HT₆ antagonists, M1 muscarinic agonists, M2 muscarinic antagonists or acetylcholinesterase inhibitors. When the compounds are used in combination with other therapeutic agents, the compounds may be administered either sequentially or simultaneously by any convenient route.

The invention thus provides, in a further aspect, a combination comprising a compound of formula (I) or a pharmaceutically acceptable derivative thereof together with a further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of formula (I) or a pharmaceutically acceptable derivative thereof is used in combination with a second therapeutic agent active against the same disease state the dose of each compound may differ from that when the compound is used alone. Appropriate doses will be readily appreciated by those skilled in the art.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusible solutions or suspensions or suppositories. Orally administrable compositions are generally preferred.

Tablets and capsules for oral administration may be in unit dose form, and may contain conventional excipients, such as binding agents, fillers, tabletting lubricants, disintegrants and acceptable wetting agents. The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents, emulsifying agents, non-aqueous vehicles (which may include edible oils), preservatives, and, if desired, conventional flavourings or colorants.

For parenteral administration, fluid unit dosage forms are prepared utilising a compound of the invention or pharmaceutically acceptable salt thereof and a sterile vehicle. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration. The compound can be sterilised by exposure to ethylene oxide before suspension in a sterile vehicle. Advantageously, a surfactant or wetting agent is included in the composition to facilitate uniform distribution of the compound.

The composition may contain from 0.1% to 99% by weight, preferably from 10 to 60% by weight, of the active material, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 1.0 to 200 mg, and such unit doses may be administered more than once a day, for example two or three a day. Such therapy may extend for a number of weeks or months.

The following Examples illustrate the preparation of compounds of the invention.

### EXAMPLE 1

### 1-(5-{[3-(Cyclopropylmethyl)-2,3,4,5-tetrahydro-1H-3-benzazepin-7-yl]oxy}-2-pyrazinyl)-2-pyrrolidinone (E1)

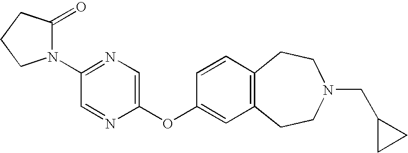

### Step 1: 5-Chloro-2-pyrazinamine

Aminopyrazine (10 g, 10.5 mmole) was dissolved in dry dimethylformamide (60 ml) and was treated with N-chlorosuccinimide (15.36 g, 11.5 mmole) under argon at 0° C. The mixture was stirred for 30 minutes and then allowed to warm to room temperature. The mixture was poured onto water and extracted with diethyl ether (×5). The diethyl ether layers were combined and evaporated in vacuo. The resulting residue was purified by column chromatography (1:9 ethyl acetate:pentane) to afford the title compound (1.40 g). ¹H NMR (CDCl₃) 8.02 (1H, s), 7.76 (1H, s), 4.61 (2H, s).

### Step 2: 2,5-Dichloropyrazine

5-Chloro-2-pyrazinamine (product of E1, step 1) (2.41 g, 18.6 mmole) was dissolved in concentrated hydrochloric acid (24 ml), cooled in an ice-acetone bath and treated with a solution of sodium nitrite (2.63 g, 38.1 mmole) in water (18 ml) dropwise over a period of 1 hour. The mixture was cooled in an ice-water bath and left to stir for 1 hour. The mixture was allowed to warm to room temperature over 1 hour, neutralised by addition of sodium hydroxide solution (2M) and extracted with dichloromethane (×4). The dichloromethane layers were combined, dried under magnesium sulfate and evaporated in vacuo. The resulting residue was purified by column chromatography (1:9 ethyl acetate:pentane) to afford the title compound (0.33 g). ¹H NMR (CDCl₃) 8.40 (2H, s).

### Step 3: 1,1-Dimethylethyl 7-[(5-chloro-2-pyrazinyl)oxy]-1,2,4,5-tetrahydro-3H-3-benzazepine-3-carboxylate

1,1-Dimethylethyl 7-hydroxy-1,2,4,5-tetrahydro-3H-3-benzazepine-3-carboxylate (Description 3 of WO 02/40471) (182 mg, 0.69 mmole) was dissolved in dry dimethylformamide (3 ml), cooled to 0° C. and treated with sodium hydride (60% in mineral oil, 29 mg, 0.72 mmole). The mixture was allowed to warm to room temperature over 60 minutes. A solution of 2,5-dichloropyrazine (product of E1, step 2) (112 mg, 0.76 mmole) in dry dimethylformamide (1 ml) was added and the mixture stirred at room temperature for 2 hours. The mixture was diluted with water (10 ml) and extracted with ethyl acetate (×2). The ethyl acetate layers were combined, dried under magnesium sulfate and evaporated in vacuo. The residue was purified by column chromatography eluting with ethyl acetate/pentane (1:4) to afford the title compound (208 mg). MS (ES+) m/e 376 [M+H]⁺.

### Step 4: 1,1-Dimethylethyl 7-{[5-(2-oxo-1-pyrrolidinyl)-2-pyrazinyl]oxy}-1,2,4,5-tetrahydro-3H-3-benzazepine-3-carboxylate

1,1-Dimethylethyl 7-[(5-chloro-2-pyrazinyl)oxy]-1,2,4,5-tetrahydro-3H-3-benzazepine-3-carboxylate (product of E1, step 3) (208 mg, 0.55 mmole), pyrrolidinone (0.08 ml, 1.1 mmole), potassium carbonate (273 mg, 1.98 mmole), copper (I) iodide (32 mg, 0.17 mmole) and N,N-dimethylethylenediamine (0.02 ml, 0.17 mmole) were added together in dry dioxane (5 ml) and heated in a microwave reactor at 150° C. for 3 hours. The mixture was diluted with water and extracted with ethyl acetate (×3). The ethyl acetate layers were combined, dried under magnesium sulfate and evaporated in vacuo. The residue was purified by column chromatography eluting with ethyl acetate/pentane (1:1) to afford the title compound (126 mg). MS (ES+) m/e 425 [M+H]⁺.

### Step 5: 1-[5-(2,3,4,5-Tetrahydro-1H-3-benzazepin-7-yloxy)-2-pyrazinyl]-2-pyrrolidinone

1,1-Dimethylethyl 7-{[5-(2-oxo-1-pyrrolidinyl)-2-pyrazinyl]oxy}-1,2,4,5-tetrahydro-3H-3-benzazepine-3-carboxylate (product of E1, step 4) (126 mg, 0.30 mmole) was dissolved in dry dichloromethane (2 ml), treated with trifluoroacetic acid (2 ml) and the resulting mixture was stirred at room temperature for 2 hours. The solvent was removed in vacuo and the residue dissolved in methanol and applied to a SCX column eluting with methanol and 2M ammonia/methanol. The basic fractions were combined and concentrated in vacuo to afford the title compound (88 mg). MS (ES+) m/e 325 [M+H]⁺.

### Step 6: 1-(5-{[3-(Cyclopropylmethyl)-2,3,4,5-tetrahydro-1H-3-benzazepin-7-yl]oxy}-2-pyrazinyl)-2-pyrrolidinone

1-[5-(2,3,4,5-Tetrahydro-1H-3-benzazepin-7-yloxy)-2-pyrazinyl]-2-pyrrolidinone (product of E1, step 5) (28 mg, 0.09 mmole) was dissolved in dry dichloromethane (2 ml), treated with cyclopropanecarboxaldehyde (0.01 ml, 0.18 mmole) and acetic acid (1 drop) and the resulting mixture stirred for 15 minutes. Sodium triacetoxyborohydride (38 mg, 0.18 mmole) was added and the mixture stirred for 18 hours. The mixture was diluted with methanol and applied to a SCX column eluting with methanol and 2M ammonia/methanol. The basic fractions were combined and concentrated in vacuo to afford the title compound (35 mg). MS (ES+) m/e 379 [M+H]⁺.

### EXAMPLE 2

### 3-(1-Methylethyl)-7-[(phenylmethyl)oxy]-2,3,4,5-tetrahydro-1H-3-benzazepine (E2)

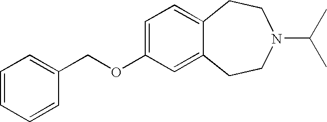

7-[(Phenylmethyl)oxy]-2,3,4,5-tetrahydro-1H-3-benzazepine (Description 2 of WO 2004056369; 120 mg, 0.47 mmol) was dissolved in dichloromethane (5 ml), treated with acetone (0.09 ml, 1.19 mmol), acetic acid (1 drop) and sodium triacetoxyborohydride (252 mg, 1.19 mmol). The resulting mixture was stirred at room temperature under argon for 2 hours. The mixture was diluted with methanol and applied to a SCX cartridge (Varian Bond-elute, 10 g) and washed with methanol, then 0.880 ammonia/methanol (1:9). The product containing fractions were concentrated in vacuo to afford the title product. MS (ES+) m/e 296 [M+H]⁺.

### EXAMPLE 3

### 3-(2-Methylpropyl)-7-[(phenylmethyl)oxy]-2,3,4,5-tetrahydro-1H-3-benzazepine (E3)

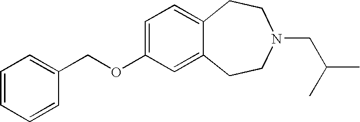

Example 3 was prepared from 7-[(phenylmethyl)oxy]-2,3,4,5-tetrahydro-1H-3-benzazepine (Description 2 of WO 2004056369) and 2-methylpropanal using the analogous method to that described for Example 2. MS (ES+) m/e 310 [M+H]⁺.

### EXAMPLE 4

### 3-Ethyl-7-[(phenylmethyl)oxy]-2,3,4,5-tetrahydro-1H-3-benzazepine (E4)

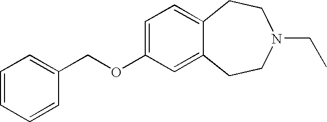

7-[(Phenylmethyl)oxy]-2,3,4,5-tetrahydro-1H-3-benzazepine (Description 2 of WO 2004056369; 200 mg, 0.79 mmol) was dissolved in 2-butanone (10 ml), treated with iodoethane (0.07 ml, 0.87 mmol), potassium carbonate (164 mg, 1.19 mmol) and potassium iodide (198 mg, 1.19 mmol) and the resulting mixture was heated under reflux for 2 hours. The mixture was allowed to cool to room temperature, diluted with water and extracted with ethyl acetate. The ethyl acetate layer was separated, dried under magnesium sulfate and concentrated in vacuo. The crude residue was purified by column chromatography eluting with a mixture of 0.880 ammonia:methanol:dichloromethane (0.5:4.5:95) to afford the title product; MS (ES+) m/e 282 [M+H]⁺.

### EXAMPLE 5

### 3-(Cyclopropylmethyl)-7-[(4-piperidinylmethyl)oxy]-2,3,4,5-tetrahydro-1H-3-benzazepine (E5)

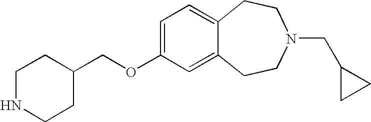

Step 1

### 2,3,4,5-Tetrahydro-1H-3-benzazepin-7-ol trifluoroacetate (salt)

1,1-Dimethylethyl 7-hydroxy-1,2,4,5-tetrahydro-3H-3-benzazepine-3-carboxylate (WO 2003068752; 10 g, 38.0 mmol) was suspended in dichloromethane (40 ml), cooled in an ice bath and treated with trifluoroacetic acid (20 ml). The mixture was stirred for 20 minutes, allowed to warm to room temperature and stirred for 1 hour. The solvent was removed in vacuo to give the title compound. ¹H NMR (DMSO) δ 9.34 (1H, brs), 8.88 (2H, brs), 6.99-6.97 (1H, d), 6.61 (1H, d), 6.56-6.54 (1H, dd), 3.20-3.05 (4H, brs), 2.94-2.91 (4H, m).

Step 2

### 3-(Cyclopropylmethyl)-2,3,4,5-tetrahydro-1H-3-benzazepin-7-ol

2,3,4,5-tetrahydro-1H-3-benzazepin-7-ol trifluoroacetate (salt) (Example 5, Step 1; 5.0 g, 18.1 mmol) was dissolved in dry dichloromethane (50 ml), cooled in an ice bath and treated with triethylamine (2.52 ml, 18.1 mmol), cyclopropanecarboxaldehyde (2.03 ml, 29.2 mmol) and sodium triacetoxyborohydride (5.77 g, 27.2 mmol) under argon. The resulting mixture was allowed to warm to ambient temperature and stirred for 18 hours. The mixture was diluted with ethyl acetate and washed with water and saturated sodium bicarbonate solution. The organic layer was separated, dried under magnesium sulfate and evaporated in vacuo to give the title compound. MS (ES+) m/e 218 [M+H]⁺.

Step 3

### 1,1-Dimethylethyl 4-({[3-(cyclopropylmethyl)-2,3,4,5-tetrahydro1H-3-benzazepin-7-yl]oxy}methyl)-1-piperidinecarboxylate

The product from Example 5, Step 2 (1.60 g, 7.37 mmol) was dissolved in dry tetrahydrofuran (20 ml) and treated with 1,1-dimethylethyl 4-(hydroxymethyl)-1-piperidinecarboxylate (J. Med. Chem, 1994, 37, 17, 2721-2734; 1.90 g, 8.84 mmol), triphenylphosphine (2.32 g, 8.84 mmol) and di-t-butylazodicarboxylate (2.03 g, 8.84 mmol). The resulting mixture was stirred at ambient temperature under argon for 18 hours. The mixture was diluted with ethyl acetate and washed with aqueous sodium hydroxide solution (2M), water and brine. The organic layer was separated, dried under magnesium sulfate and evaporated in vacuo. The residue was purified by column chromatography eluting with ethyl acetate/hexane (1:4) to give the title compound. MS (ES+) m/e 415 [M+H]⁺.

Step 4

### 3-(Cyclopropylmethyl)-7-[(4-piperidinylmethyl)oxy]-2,3,4,5-tetrahydro-1H-3-benzazepine

The product from example 5, step 3 (1.19 g, 2.87 mmol) was dissolved in dichloromethane (10 ml), treated with trifluoroacetic acid (3 ml) and stirred at room temperature under argon for 2 hours. The solvent was removed in vacuo and the residue dissolved in methanol and passed down a SCX cartridge (Varian Bond-elute, 10 g) eluting with methanol followed by 0.880 ammonia/methanol (1:9). The basic fractions were combined and concentrated in vacuo. The residue was purified by column chromatography eluting with 0.880 ammonia/methanol/dichloromethane (0.5:4.5:95) to give the title compound. MS(ES+) m/e 315 [M+H]⁺.

### EXAMPLE 6

### 4-{[4-({[3-(Cyclopropylmethyl)-2,3,4,5-tetrahydro-1H-3-benzazepin-7-yl]oxy}methyl)-1-piperidinyl]carbonyl}benzonitrile (E6)

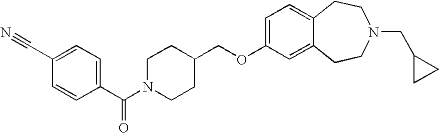

3-(Cyclopropylmethyl)-7-[(4-piperidinylmethyl)oxy]-2,3,4,5-tetrahydro-1H-3-benzazepine (E5; 100 mg, 0.32 mmol) was dissolved in dichloromethane (5 ml), treated with 4-cyanobenzoic acid (118 mg, 0.80 mmol), N,N′-dcyclohexylcarbodiimide (165 mg, 0.80 mmol) and 1-hydrocybenzotriazole (108 mg, 0.80 mmol) and resulting mixture stirred at room temperature under argon for 18 hours. The mixture was diluted with methanol and applied to a SCX cartridge (Varian Bond-elute, 10 g) and washed with methanol, then 0.880 ammonia/methanol (1:9). The product containing fractions were concentrated in vacuo. The residue was purified by column chromatography eluting with 0.880 ammonia/methanol/dichloromethane (0.5:4.5:95) to give the title compound. MS(ES+) m/e 444 [M+H]⁺.

### EXAMPLE 7-9 (E7-E9)

Examples 7-9 were prepared from 3-(cyclopropylmethyl)-7-[(4-piperidinylmethyl)oxy]-2,3,4,5-tetrahydro-1H-3-benzazepine (E5) and the appropriate carboxylic acid using an analogous method to that described for Example 6 (see table).

| | | LC/MS |
|---|---|---|
| | | (M + |
| Example | Carboxylic acid | H⁺) |
| 3-(Cyclopropylmethyl)-7-[({1-[(4- | 4-Fluorobenzoic acid | 437 |
| fluorophenyl)carbonyl]-4- | | |
| piperidinyl}methyl)oxyl]-2,3,4,5- | | |
| tetrahydro-1H-3-benzazepine (E7) | | |
| 7-({[1-(Cyclopropylcarbonyl)-4- | Cyclopropanecarboxylic | 383 |
| piperidinyl]methyl}oxy)-3- | acid | |
| (cyclopropylmethyl)-2,3,4,5- | | |
| tetrahydro-1H-3-benzazepine (E8) | | |
| 3-(Cyclopropylmethyl)-7-({[1- | Tetrahydro-2H-pyran-4- | 427 |
| (tetrahydro-2H-pyran-4-ylcarbonyl)- | carboxylic acid | |
| 4-piperidinyl]methyl}oxy)-2,3,4,5- | | |
| tetrahydro-1H-3-benzazepine (E9) | | |

### EXAMPLE 10

### 1-(6-{[3-(1-Methylethyl)-2,3,4,5-tetrahydro-1H-3-benzazepin-7-yl]oxy}-3-pyridinyl)-2-pyrrolidinone (E10)

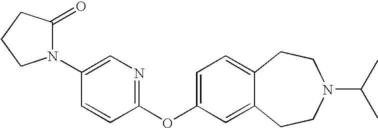

### Step 1: 1,1-Dimethylethyl 7-[(5-bromo-2-pyridinyl)oxy]-1,2,4,5-tetrahydro-3H-3-benzazepine-3-carboxylate

1,1-Dimethylethyl 7-hydroxy-1,2,4,5-tetrahydro-3H-3-benzazepine-3-carboxylate (Description 3 of WO 02/40471) (50 g, 0.21 mole) was dissolved in dry N-methyl pyrrolidinone (350 ml) and treated with sodium hydride (60% in mineral oil, 8.9 g, 0.22 mole). The mixture was stirred at room temperature for 3 hours. 5-bromo-2-chloropyridine (44.8 g, 0.23 mole) was added and the mixture stirred at 95° C. for 18 hours. The mixture was diluted with water and the resulting solid collected by filtration. This was dissolved in ethyl acetate dried (sodium sulphate) and evaporated under reduced pressure. The residue was purified by column chromatography eluting with ethyl acetate/pentane (1:9) to afford the title compound MS (AP+) m/e 419 & 420 [M+H]⁺.

### Step 2: 1,1-Dimethylethyl 7-{[5-(2-oxo-1-pyrrolidinyl)-2-pyridinyl]oxy}-1,2,4,5-tetrahydro-3H-3-benzazepine-3-carboxylate

Copper (I) iodide (2.27 g, 12 mmol) was added to a stirring mixture of 1,1-dimethylethyl 7-[(5-bromo-2-pyridinyl)oxy]-1,2,4,5-tetrahydro-3H-3-benzazepine-3-carboxylate (E10, Step 1) (50 g, 0.12 M), 2-pyrrolidinone (20.3 g, 0.24 mol), potassium carbonate (59 g 0.43 mol) and 1,2-ethanediamine (1.01 g, 12 mmol) in 1,4-dioxan (400 ml) and the mixture heated at reflux for 18 hours. The mixture was allowed to cool and filtered through Celite. The filtrate was evaporated and the residue was purified by column chromatography eluting with ethyl acetate/pentane (1:1) to afford the title compound MS (AP+) m/e 424 [M+H]⁺.

### Step 3: 1-[6-(2,3,4,5-Tetrahydro-1H-3-benzazepin-7-yloxy)-3-pyridinyl]-2-pyrrolidinone

A solution of 1,1-dimethylethyl 7-{[5-(2-oxo-1-pyrrolidinyl)-2-pyridinyl]oxy}-1,2,4,5-tetrahydro-3H-3-benzazepine-3-carboxylate (E10, Step 2) (13.7 g, 32.4 mmol) in dichloromethane (80 ml) was added drop wise to 4M hydrogen chloride in 1,4-dioxan (81 ml, 0.32 M) and the mixture stirred for 90 minutes. The resulting solid was collected by filtration and washed with dichloromethane. The solid was dissolved in water and basified using 12.5 M sodium hydroxide solution. This solution was extracted with dichloromethane, the extracts were combined, dried (sodium sulphate) and evaporated under reduced pressure to afford the title compound MS (AP+) m/e 324 [M+H]⁺.

### Step 4: 1-(6-{[3-(1-Methylethyl)-2,3,4,5-tetrahydro-1H-3-benzazepin-7-yl]oxy}-3-pyridinyl)-2-pyrrolidinone

A mixture of 1-[6-(2,3,4,5-tetrahydro-1H-3-benzazepin-7-yloxy)-3-pyridinyl]-2-pyrrolidinone (E10, step 3) (150 mg, 0.46 mmol), acetone (0.17 ml, 2.3 mmol) sodium triacetoxyborohydride (196 mg, 0.92 mmol) and glacial acetic acid (50 μl) in dichloromethane (5 ml) was stirred at room temperature for 3 hours. The crude mixture was purified on an SCX ion exchange cartridge eluting with methanol and then 2M ammonia in methanol. The basic fractions were combined, evaporated under reduced pressure and the residue purified by chromatography on silica eluting with 97-3 dichloromethane-2M ammonia in methanol to afford the title compound as a colourless gum. MS (AP+) m/e 366 [M+H]⁺.

### EXAMPLES 11-16 (E11-E16)

5 Examples E11-E16 were prepared from 1-[6-(2,3,4,5-Tetrahydro-1H-3-benzazepin-7-yloxy)-3-pyridinyl]-2-pyrrolidinone (E10, step 3) and the ketones shown in the table below using the same method as for E10, step 4:

| | | Mass |
|---|---|---|
| Example | Ketone | Spectrum |
| 1-(6-{[3-(2-Methylpropyl)-2,3,4,5- | 2-Methylpropanal | MS (AP+), m/e |
| tetrahydro-1H-3-benzazepin-7- | | 380 [M + H]⁺. |
| yl]oxy}-3-pyridinyl)-2-pyrrolidinone | | |
| (E11) | | |
| 1-(6-{[3-(2,2-Dimethylpropyl)- | 2,2- | MS (AP+), m/e |
| 2,3,4,5-tetrahydro-1H-3- | Dimethylpropanal | 394 [M + H]⁺. |
| benzazepin-7-yl]oxy}-3-pyridinyl)- | | |
| 2-pyrrolidinone (E12) | | |
| 1-(6-{[3-(Cyclopropylmethyl)- | Cyclopropane | MS (AP+), m/e |
| 2,3,4,5-tetrahydro-1H-3- | carbaldehyde | 378 [M + H]⁺. |
| benzazepin-7-yl]oxy}-3-pyridinyl)- | | |
| 2-pyrrolidinone (E13) | | |
| 1-{6-[(3-Ethyl-2,3,4,5-tetrahydro-1H- | Acetaldehyde | MS (AP+), m/e |
| 3-benzazepin-7-yl)oxy]-3-pyridinyl}- | | 378 [M + H]⁺. |
| 2-pyrrolidinone (E14) | | |
| 1-(6-{[3-(1-Methylpropyl)-2,3,4,5- | 2-Butanone | MS (AP+), m/e |
| tetrahydro-1H-3-benzazepin-7- | | 380 [M + H]⁺. |
| yl]oxy}-3-pyridinyl)-2-pyrrolidinone | | |
| (E15) | | |
| 1-(6-{[3-(Cyclobutylmethyl)-2,3,4,5- | Cyclobutane | MS (AP+), m/e |
| tetrahydro-1H-3-benzazepin-7- | carbaldehyde | 392 [M + H]⁺. |
| yl]oxy}-3-pyridinyl)-2-pyrrolidinone | | |
| (E16) | | |

### EXAMPLE 17

### 3-(Cyclopropylmethyl)-7-{[5-(3-methyl-1,2,4-oxadiazol-5-yl)-2-pyridinyl]oxy}-2,3,4,5-tetrahydro-1H-3-benzazepine (E17)

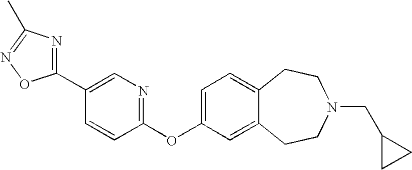

### Step 1: 1,1-Dimethylethyl 7-({5-[(methyloxy)carbonyl]-2-pyridinyl}oxy)-1,2,4,5-tetrahydro-3H-3-benzazepine-3-carboxylate

1,1-Dimethylethyl 7-hydroxy-1,2,4,5-tetrahydro-3H-3-benzazepine-3-carboxylate (Description 3 of WO 02/40471) (3 g, 11.4 mmole) was dissolved in dry dimethylformamide (20 ml), cooled to 0° C. and treated with sodium hydride (60% in mineral oil, 540 mg, 13.7 mmole). The mixture was allowed to warm to room temperature over 60 minutes. Methyl 6-chloro-3-pyridinecarboxylate (2.4 g, 13.7 mmole) was added and the mixture stirred at 100° C. for 18 hours. The mixture was diluted with water and extracted with ethyl acetate. The ethyl acetate layers were combined, dried (sodium sulphate) and evaporated in vacuo. The residue was purified by column chromatography eluting with ethyl acetate/pentane (1:4) to afford the title compound. MS (AP+) m/e 399 [M+H]⁺.

### Step 2: 6-[(3-{[(1,1-Dimethylethyl)oxy]carbonyl}-2,3,4,5-tetrahydro-1H-3-benzazepin-7-yl)oxy]-3-pyridinecarboxylic acid

2 M Sodium hydroxide solution (14 ml, 28 mmol) was added to a solution of 1,1-dimethylethyl 7-({5-[(methyloxy)carbonyl]-2-pyridinyl}oxy)-1,2,4,5-tetrahydro-3H-3-benzazepine-3-carboxylate (E17, Step 1). (3.7 g, 9.3 mmol) in acetone (35 ml) and the mixture stirred at room temperature for 18 hours. The mixture was acidified using 2 M hydrochloric acid and extracted with ethyl acetate. The extracts were dried (sodium sulphate) and evaporated to afford the title compound MS (AP+) m/e 399 [M+H]⁺.

### Step 3: 1,1-Dimethylethyl 7-{[5-(3-methyl-1,2,4-oxadiazol-5-yl)-2-pyridinyl]oxy}-1,2,4,5-tetrahydro-3H-3-benzazepine-3-carboxylate

A mixture of 6-[(3-{[(1,1-dimethylethyl)oxy]carbonyl}-2,3,4,5-tetrahydro-1H-3-benzazepin-7-yl)oxy]-3-pyridinecarboxylic acid (E17, Step 2) (3.1 g, 8 mmol) and 1,1′-(oxomethanediyl)bis-1H-imidazole (1.42 g, 8.8 mmol) in tetrahydrofuran (30 ml) was heated at reflux for 90 minutes. N-Hydroxyethanimidamide was added and the mixture stirred at reflux for 3 days. The reaction mixture was diluted with ethyl acetate, washed with water and 2M sodium hydroxide solution, dried (sodium sulphate) and evaporated under reduced pressure. The residue was purified by column chromatography eluting with ethyl acetate/pentane (1:4) to afford the title compound. MS (AP+) m/e 423 [M+H]⁺.

### Step 4: 7-{[5-(3-Methyl-1,2,4-oxadiazol-5-yl)-2-pyridinyl]oxy}-2,3,4,5-tetrahydro-1H-3-benzazepine

A solution of 1,1-dimethylethyl 7-{[5-(3-methyl-1,2,4-oxadiazol-5-yl)-2-pyridinyl]oxy}-1,2,4,5-tetrahydro-3H-3-benzazepine-3-carboxylate (E17, Step 3) (1.68 g, 4 mmol) in dichloromethane (15 ml) was treated with trifluoroacetic acid (15 ml) and the mixture stirred for 1 hour. The solvent was removed by evaporation under reduced pressure and the residue was purified on an SCX ion exchange cartridge eluting with methanol and then 2M ammonia in methanol. The basic fractions were combined and evaporated to afford the title compound ¹H NMR (CDCl₃) δ 8.93 (H, m), 8.34 (H, m), 7.16 (H, m), 7.03 (H, m), 6.90 (2H, m), 2.91-3.00 (8H, m), 2.47 (3H, s)

### Step 5: 3-(Cyclopropylmethyl)-7-{[5-(3-methyl-1,2,4-oxadiazol-5-yl)-2-pyridinyl]oxy}-2,3,4,5-tetrahydro-1H-3-benzazepine

This compound was synthesised using 7-{[5-(3-Methyl-1,2,4-oxadiazol-5-yl)-2-pyridinyl]oxy}-2,3,4,5-tetrahydro-1H-3-benzazepine (E17, step 4) and cyclopropane carbaldehyde using the method described in Example 10, step 4 MS (AP+) m/e 377 [M+H]⁺.

### EXAMPLE 18

### 1-(4-{[3-(Cyclopropylmethyl)-2,3,4,5-tetrahydro-1H-3-benzazepin-7-yl]oxy}phenyl)-3-methyl-2-imidazolidinone (E18)

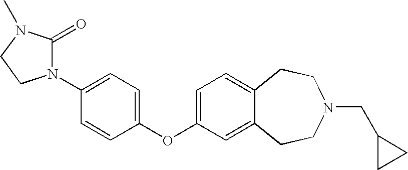

### Step 1: 3-(Cyclopropylmethyl)-7-[(4-iodophenyl)oxy]-2,3,4,5-tetrahydro-1H-3-benzazepine

A mixture of 3-(cyclopropylmethyl)-2,3,4,5-tetrahydro-1H-3-benzazepin-7-ol (E5, step 2) (242 mg, 1.1 mmol), 1-fluoro-4-iodobenzene (298 mg, 1.34 mmol) and caesium carbonate (436 mg, 1.34 mmol) in dimethylformamide (5 ml) was heated at 200° C. in a microwave at high absorption for 45 minutes. The crude mixture was purified on an SCX ion exchange cartridge eluting with methanol and then 2M ammonia in methanol. The basic fractions were combined, evaporated under reduced pressure and the residue purified by chromatography on silica eluting with 99-1 dichloromethane-2M ammonia in methanol to afford the title compound as a colourless gum. MS (AP+) m/e 420 [M+H]⁺.

### Step 2: 1-(4-{[3-(Cyclopropylmethyl)-2,3,4,5-tetrahydro-1H-3-benzazepin-7-yl]oxy}phenyl)-3-methyl-2-imidazolidinone

A mixture of 3-(cyclopropylmethyl)-7-[(4-iodophenyl)oxy]-2,3,4,5-tetrahydro-1H-3-benzazepine (E18, Step 1), (168 mg, 0.4 mmol), 1-methyl-2-imidazolidinone (80 mg, 0.8 mmol)), potassium carbonate (200 mg, 1.44 mmol) copper (I) iodide (23 mg, 0.12 mmol) and 1,2-ethanediamine (11 mg, 0.12 mmol) in 1,4-dioxan (5 ml) and the mixture heated at 140° C. in a microwave at high absorption for 105 minutes. The mixture was diluted with water and extracted with ethyl acetate. The ethyl acetate layers were combined, dried (sodium sulphate) and evaporated in vacuo. The residue was purified by column chromatography eluting with 97-3 dichloromethane-2M ammonia in methanol to afford the title compound. MS (AP+) m/e 392 [M+H]⁺.

### EXAMPLE 19

### 3-(Cyclopropylmethyl)-7-[(phenylmethyl)oxy]-2,3,4,5-tetrahydro-1H-3-benzazepine (E19)

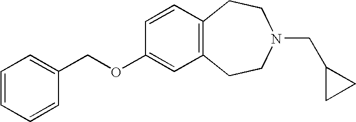

### Step 1: 1,1-Dimethylethyl 7-[(phenylmethyl)oxy]-1,2,4,5-tetrahydro-3H-3-benzazepine-3-carboxylate

1,1-Dimethylethyl 7-hydroxy-1,2,4,5-tetrahydro-3H-3-benzazepine-3-carboxylate (Description 3 in WO 02/40471) (790 mg, 3 mmol) and potassium carbonate (1.24 g, 9 mmol) were suspended in 2-butanone (20 ml). Benzyl bromide (536 uL, 4.5 mmol) was added to the mixture and heated at reflux for 48 hours. The solids were filtered and the filtrate concentrated in vacuo. The residue was purified by column chromatography, eluting with a gradient of hexane to 30% ethyl acetate in hexane to afford the title compound. ¹H NMR (CDCl₃) δ 7.38 (5H, m), 7.03 (1H, d, J 8.0 Hz), 6.65 (2H, m), 5.03 (2H, s), 3.49 (4H, m), 2.84 (4H, m), 2.84 (4H, m), 1.48 (9H, s).

### Step 2: 7-[(Phenylmethyl)oxy]-2,3,4,5-tetrahydro-1H-3-benzazepine

The product of example 19, step 1 (1.06 g, ca. 3 mmol) was dissolved in dichloromethane (15 ml) at 0° C. and treated with trifluoroacetic acid (15 ml). The solution was stirred at room temperature for 2 hours and solvents removed in vacuo. The residue was passed through a SCX cartridge (Varian, 10 g), washing with methanol and eluting products with 10% 0.880 ammonia in methanol. The ammoniacal solution was concentrated in vacuo and the residue purified by column chromatography, eluting with a gradient of dichloromethane to 1:9:190 0.880 ammonia:ethanol:dichloromethane, to afford the title compound; MS (ES+) m/e 254 [M+H]⁺.

### Step 3: 3-(Cyclopropylmethyl)-7-[(phenylmethyl)oxy]-2,3,4,5-tetrahydro-1H-3-benzazepine

The crude product of example 19 step 2 (265 mg, 0.75 mmol), cyclopropanecarboxaldehyde (168 μL, 2.3 mmol) and (polystyrylmethyl)trimethylammonium cyanoborohydride (750 mg, 4 mmol/g loading, 3 mmol) were stirred at room temperature for 18 hours in 5% acetic acid in methanol (5 ml). The solution was then treated was stirred at room temperature for 18 hours. The reaction mixture was applied to an SCX column (Varian bond-elute, 10 g) eluting with methanol followed by a mixture of 0.880 ammonia/methanol (1:9). The basic fractions were combined and concentrated in vacuo giving the title compound. MS(ES+) m/e 308 [M+H]⁺.

### EXAMPLE 20

### 7-[(3-Cyclohexylpropyl)oxy]-3-(cyclopropylmethyl)-2,3,4,5-tetrahydro-1H-3-benzazepine (E20)

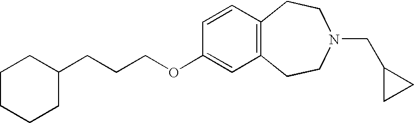

The title compound was prepared from 1,1-Dimethylethyl 7-hydroxy-1,2,4,5-tetrahydro-3H-3-benzazepine-3-carboxylate (Description 3 in WO 02/40471) and 1-bromo-3-cyclohexyl propane using an analogous 3 step procedure to that used in example 19; MS(ES+) m/e 348 [M+H]⁺.

### EXAMPLE 21

### 3-(Cyclopropylmethyl)-7-(phenyloxy)-2,3,4,5-tetrahydro-1H-3-benzazepine (E21)

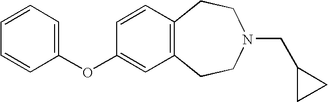

### Step 1. 1,1-Dimethylethyl 7-(phenyloxy)-1,2,4,5-tetrahydro-3H-3-benzazepine-3-carboxylate

Sodium hydride (60% dispersion in mineral oil) (192 mg, 4.8 mmol) was suspended in pyridine (2.5 ml) and cooled to 0° C. The mixture was treated with 1,1-Dimethylethyl 7-hydroxy-1,2,4,5-tetrahydro-3H-3-benzazepine-3-carboxylate (Description 3 in WO 02/40471) (1.05 g, 4 mmol) dissolved in pyridine (7.5 ml) and stirred at this temperature for 15 minutes. Copper(I) bromide (803 mg, 5.6 mmol) was added and stirring continued at room temperature for 30 minutes whereupon iodobenzene (895 uL, 8 mmol) dissolved in pyridine (5 ml) was added. The mixture was heated at reflux overnight, cooled, filtered and concentrated in vacuo. The residue was purified by column chromatography eluting with a gradient of 5% to 10% ethyl acetate in hexane to afford the title product; MS(ES+) m/e 294 [M+H]⁺.

### Step 2. 7-(Phenyloxy)-2,3,4,5-tetrahydro-1H-3-benzazepine

The title compound was prepared from 1,1-dimethylethyl 7-(phenyloxy)-1,2,4,5-tetrahydro-3H-3-benzazepine-3-carboxylate (Example 21, step 1) using an analogous procedure to that used in example 19, Step 2. MS(ES+) m/e 240 [M+H]⁺.

### Step 3. 3-(Cyclopropylmethyl)-7-(phenyloxy)-2,3,4,5-tetrahydro-1H-3-benzazepine

The title compound was prepared from 7-(phenyloxy)-2,3,4,5-tetrahydro-1H-3-benzazepine (Example 21, step 2) and cyclopropane carboxaldehyde using an analogous procedure to that used in example 19, Step 3. MS(ES+) m/e 294 [M+H]⁺.

### EXAMPLE 22

### Ethyl 4-{[3-(cyclopropylmethyl)-2,3,4,5-tetrahydro-1H-3-benzazepin-7-yl]oxy}benzoate (E22)

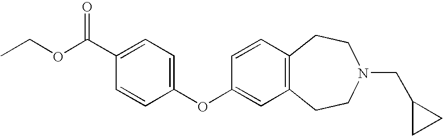

The title compound was prepared from 1,1-Dimethylethyl 7-hydroxy-1,2,4,5-tetrahydro-3H-3-benzazepine-3-carboxylate (Description 3 in WO 02/40471) and ethyl 4-bromo benzoate using an analogous 3 step procedure to that used as example 19 MS(ES+) m/e 366 [M+H]⁺.

### EXAMPLE 23

### 6-{[3-(Cyclopropylmethyl)-2,3,4,5-tetrahydro-1H-3-benzazepin-7-yl]oxy}-N-methyl-3-pyridinecarboxamide (E23)

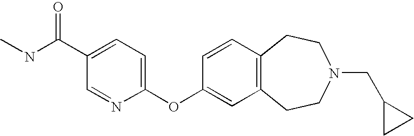

### Step 1: 6-Chloro-N-methyl-3-pyridinecarboxamide

2M Methylamine in tetrahydrofuran (100 ml, 200 mol) was cooled to 0° C. and 6-chloronicotinoyl chloride (10.6 g, 60 mmol) dissolved in dichloromethane (30 ml) was added dropwise. The solution was stirred to room temperature overnight and concentrated in vacuo. The residue was partitioned between dichloromethane and water, the aqueous phase was extracted with dichloromethane (×3). The combined extracts were washed with water, saturated brine and dried over anhydrous magnesium sulphate. The filtrate was concentrated to a crude solid that was used without further purification. (ES+) m/e 171 [M+H]⁺.

### Step 2. 1,1-Dimethylethyl 7-({5-[(methylamino)carbonyl]-2-pyridinyl}oxy)-1,2,4,5-tetrahydro-3H-3-benzazepine-3-carboxylate

1,1-Dimethylethyl 7-hydroxy-1,2,4,5-tetrahydro-3H-3-benzazepine-3-carboxylate (Description 3 in WO 02/40471) (8.7 g, 33 mmol) was dissolved in tert-butanol (70 ml) and treated with potassium tert-butoxide (4 g, 36 mmol) for 30 minutes. The product of example 23, step 1 (5.1 g, 30 mmol) was added and the mixture heated at reflux for 20 hours. The mixture was concentrated in vacuo and the residue partitioned between ethyl acetate and water. The organic phase was washed with brine, dried over anhydrous magnesium sulphate and concentrated in vacuo. The residue was purified by column chromatography eluting with a gradient of 2.5 to 5% methanol in dichloromethane to afford the title product; MS(ES+) m/e 398 [M+H]⁺.

### Step 3. N-methyl-6-(2,3,4,5-tetrahydro-1H-3-benzazepin-7-yloxy)-3-pyridinecarboxamide

The product of example 23, step 2 (3.98 g, 10 mmol) was dissolved in dioxane (40 ml) and 4M hydrogen chloride in dioxane (25 ml, 100 mmol). The mixture was stirred at room temperature for 5 hours, and concentrated in vacuo. The resulting solid was dissolved in water and basified with 0.880 ammonia solution. The aqueous phase was extracted with dichloromethane (×3), combined extracts washed with saturated brine, dried over anhydrous sodium sulphate, and concentrated to afford crude product that was used without subsequent purification. MS(ES+) m/e 298 [M+H]⁺.

### Step 4. 6-{[3-(Cyclopropylmethyl)-2,3,4,5-tetrahydro-1H-3-benzazepin-7-yl]oxy}-N-methyl-3-pyridinecarboxamide

The title compound was prepared from N-methyl-6-(2,3,4,5-tetrahydro-1H-3-benzazepin-7-yloxy)-3-pyridinecarboxamide (example 23, step 3) and cyclopropane carboxaldehyde using an analogous procedure to that used in example 19, Step 3. MS(ES+) m/e 352 [M+H]⁺.

### EXAMPLE 24

5-{[3-(Cyclopropylmethyl)-2,3,4,5-tetrahydro-1H-3-benzazepin-7-yl]oxy}-N-methyl-2-pyrazinecarboxamide (E24)

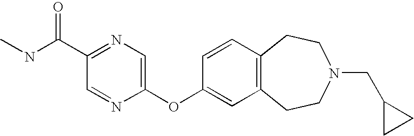

### Step 1: 1,1-Dimethylethyl 7-({5-[(methyloxy)carbonyl]-2-pyrazinyl}oxy)-1,2,4,5-tetrahydro-3H-3-benzazepine-3-carboxylate

1,1-Dimethylethyl 7-hydroxy-1,2,4,5-tetrahydro-3H-3-benzazepine-3-carboxylate (Description 3 of WO 02/40471) (40 g, 0.15 mole) was dissolved in dry dimethylformamide (200 ml) and treated with sodium hydride (60% in mineral oil, 6.4 g, 0.16 mole). The mixture was stirred at room temperature for 1 hour and then cooled in ice. Methyl 5-chloro-2-pyrazinecarboxylate (31.2 g, 0.18 mole) was added and the mixture stirred at room temperature for 18 hours. The mixture was poured into ice/water and the resulting solid collected by filtration. This was dissolved in ethyl acetate, dried (sodium sulphate) and evaporated under reduced pressure. The residue was purified by column chromatography eluting with ethyl acetate/pentane (1:2) to afford the title compound ¹H NMR (CDCl₃) δ 8.84 (H, s), 8.48 (H, s), 7.18 (H, m), 6.94 (2H, m), 4.01 (3H, s), 3.58 (4H, m), 2.92 (4H, m), 1.49 (9H, s).

### Step 2: 5-[(3-{[(1,1-Dimethylethyl)oxy]carbonyl}-2,3,4,5-tetrahydro-1H-3-benzazepin-7-yl)oxy]-2-pyrazinecarboxylic acid

2 M Sodium hydroxide solution (110 ml, 0.22 moles) was added to stirring solution of 1,1-dimethylethyl 7-({5-[(methyloxy)carbonyl]-2-pyrazinyl}oxy)-1,2,4,5-tetrahydro-3H-3-benzazepine-3-carboxylate (E24, Step 1) (29.4 g, 73.6 mmol) in acetone (480 ml) and the mixture stirred at room temperature for 30 minutes. The mixture was acidified using 2M hydrochloric acid and poured into water. The resulting solid was collected by filtration, washed with water and dissolved in ethyl acetate. This was dried (sodium sulphate) and evaporated under reduced pressure to afford the title compound MS (AP+) m/e 384 [M−H].

### Step 3: 1,1-Dimethylethyl 7-({5-[(methylamino)carbonyl]-2-pyrazinyl}oxy)-1,2,4,5-tetrahydro-3H-3-benzazepine-3-carboxylate

1,1′-(Oxomethanediyl)bis-1H-imidazole (16.6 g, 0.10 mole) was added to a stirring solution of 5-[(3-{[(1,1-dimethylethyl)oxy]carbonyl}-2,3,4,5-tetrahydro-1H-3-benzazepin-7-yl)oxy]-2-pyrazinecarboxylic acid (E24, Step 2) (37.5 g, 97 mmol) in dichloromethane (400 ml) and the mixture stirred for 18 hours. 2 M Methylamine in tetrahydrofuran (100 ml, 0.2 mole) was added and the mixture stirred at room temperature for 2 hours The solvent was removed by evaporation and the residue was purified by column chromatography eluting with ethyl acetate/chloroform (1:1) to afford the title compound MS (AP+) m/e 399 [M−H].

### Step 4: N-methyl-5-(2,3,4,5-tetrahydro-1H-3-benzazepin-7-yloxy)-2-pyrazinecarboxamide

A solution of 1,1-dimethylethyl 7-({5-[(methylamino)carbonyl]-2-pyrazinyl}oxy)-1,2,4,5-tetrahydro-3H-3-benzazepine-3-carboxylate (E24, Step 3) (44.3 g, 0.11 mole) in dichloromethane (800 ml) was added drop wise to 4M hydrogen chloride in 1,4-dioxan (270 ml, 1.1 mole) and the mixture stirred for 90 minutes. The resulting solid was collected by filtration and washed with dichloromethane. The solid was dissolved in water and basified using 2 M sodium carbonate solution. The mixture was filtered through Celite and the pad washed with hot methanol. The filtrates were evaporated to afford the title compound MS (AP+) m/e 299 [M−H].

### Step 5: 5-{[3-(Cyclopropylmethyl)-2,3,4,5-tetrahydro-1H-3-benzazepin-7-yl]oxy}-N-methyl-2-pyrazinecarboxamide

The title compound was prepared from example 24, step 4 and cyclopropane carboxaldehyde using an analogous procedure to that used in example 19, Step 3. MS(ES+) m/e 353 [M+H]⁺.

### EXAMPLE 25

### 1,1-Dimethylethyl 4-{[3-(cyclopropylmethyl)-2,3,4,5-tetrahydro-1H-3-benzazepin-7-yl]oxy}-1-piperidinecarboxylate (E25)

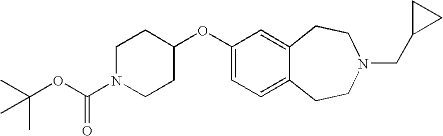

A mixture of 3-(cyclopropylmethyl)-2,3,4,5-tetrahydro-1H-3-benzazepin-7-ol (GB1268243) (0.75 g, 3.46 mmol), 1,1-dimethylethyl 4-hydroxy-1-piperidinecarboxylate (0.83 g, 4.14 mmol), triphenylphosphine (1.1 g, 4.14 mmol) and bis(1,1-dimethylethyl) 1,2-diazenedicarboxylate (0.95 g, 4.14 mmol) in tetrahydrofuran (20 ml) was stirred at room temperature for 18 hours. The mixture was then applied to an SCX ion exchange cartridge and washed with methanol and then a 2M ammonia in methanol solution. The basic fractions were reduced and the residue purified on silica gel eluting with a mixture of 2M ammonia in mathanol:dichloromethane (5:95) to afford the title product (E25) (0.8 g). MS (ES+) m/e 401 [M+H]⁺.

### EXAMPLE 26

### 3-(Cyclopropylmethyl)-7-(4-piperidinyloxy)-2,3,4,5-tetrahydro-1H-3-benzazepine (E26)

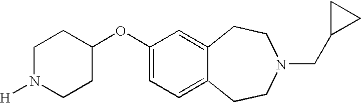

The title product (E26) was prepared from 1,1-dimethylethyl 4-{[3-(cyclopropylmethyl)-2,3,4,5-tetrahydro-1H-3-benzazepin-7-yl]oxy}-1-piperidinecarboxylate (E25) (0.8 g, 2 mmol) using the method described for Example 1, Step 5; m/e 301 [M+H]⁺.

### EXAMPLE 27

### 4-[(4-{[3-(Cyclopropylmethyl)-2,3,4,5-tetrahydro-1H-3-benzazepin-7-yl]oxy}-1-piperidinyl)carbonyl]benzonitrile (E27)

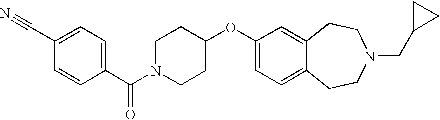

The title product (E27) was prepared from 3-(cyclopropylmethyl)-7-(4-piperidinyloxy)-2,3,4,5-tetrahydro-1H-3-benzazepine (E26) (0.1 g, 0.332 mmol) using the method described for Example 6; m/e 430 [M+H]⁺.

### EXAMPLE 28

### 1-(5-{[3-(Cyclopropylmethyl)-2,3,4,5-tetrahydro-1H-3-benzazepin-7-yl]oxy}-2-pyridinyl)-2-pyrrolidinone (E28)

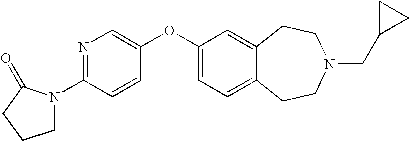

### Step 1: 1,1-Dimethylethyl 7-[(6-chloro-3-pyridinyl)oxy]-1,2,4,5-tetrahydro-3H-3-benzazepine-3-carboxylate

A mixture of (3-{[(1,1-dimethylethyl)oxy]carbonyl}-2,3,4,5-tetrahydro-1H-3-benzazepin-7-yl)boronic acid (3.02 g, 10.38 mmol) (WO2004056369), 6-chloro-3-pyridinol (1.61 g, 12.46 mmol), copper(II) acetate (5.64 g, 31.1 mmol), pyridine (4.19 ml) and molecular sieves (6.0 g, 4A) in dichloromethane was stirred at room temperature for 1 week. The solvent was removed in vacuo and the residue dissolved in ethyl acetate and washed with saturate sodium bicarbonate solution and then brine. The organic layer was then dried (MgSO₄), reduced in vacuo and purified by column chromatography eluting with a mixture of ethyl acetate:pentane (1:4) to furnish the product; m/e 375 [M+H]⁺.

### Step 2: 1,1-Dimethylethyl 7-{[6-(2-oxo-1-pyrrolidinyl)-3-pyridinyl]oxy}-1,2,4,5-tetrahydro-3H-3-benzazepine-3-carboxylate

A mixture of 1,1-dimethylethyl 7-[(6-chloro-3-pyridinyl)oxy]-1,2,4,5-tetrahydro-3H-3-benzazepine-3-carboxylate (E28, step 1; 106 mg, 0.28 mmol), pyrrolidinone (0.04 ml, 0.57 mmol), tris(dibenzylideneacetone)dipalladium (0) (0.013 g, 0.014 mmol), xantphos (24 mg, 0.042 mmol), cesium carbonate (137 mg,0.42 mmol), dioxane (3 ml) was heated in a microwave for 20 minutes at 140° C. The mixture was then filtered through celite, reduced in vacuo and chromatographed on silica gel eluting with a mixture of ethyl acetate:pentane (1:1) furnish the product; m/e 424 [M+H]⁺.

### Step 3: 1-[5-(2,3,4,5-Tetrahydro-1H-3-benzazepin-7-yloxy)-2-pyridinyl]-2-pyrrolidinone

The product was prepared from 1,1-dimethylethyl 7-{[6-(2-oxo-1-pyrrolidinyl)-3-pyridinyl]oxy}-1,2,4,5-tetrahydro-3H-3-benzazepine-3-carboxylate (E28, step 2) using the method described in Example 1 Step 5; m/e 392 [M+H]⁺.

### Step 4: 1-(5-{[3-(Cyclopropylmethyl)-2,3,4,5-tetrahydro-1H-3-benzazepin-7-yl]oxy}-2-pyridinyl)-2-pyrrolidinone

To a stirred solution of 1-[5-(2,3,4,5-tetrahydro-1H-3-benzazepin-7-yloxy)-2-pyridinyl]-2-pyrrolidinone (E28, step 3; 0.05 g, 0.155 mmol) was dissolved in dry dichloromethane (2 ml), treated with cyclopropanecarboxaldehyde (0.022 g, 0.31 mmol) and acetic acid (1 drop) and the resulting mixture stirred for 15 minutes. Sodium triacetoxyborohydride (0.055 g, 0.31 mmole) was added and the mixture stirred for 18 hours. The mixture was diluted with methanol and applied to a SCX column eluting with methanol and 2M ammonia/methanol. The basic fractions were combined and concentrated in vacuo and the residue purified on silica gel eluting with a mixture of 2M ammonia in mathanol:dichloromethane (5:95) to afford the title product (E28) (50 mg). MS (ES+) m/e 378 [M+H]⁺.

### EXAMPLE 29

### 1-(5-{[3-(2-Methylpropyl)-2,3,4,5-tetrahydro-1H-3-benzazepin-7-yl]oxy}-2-pyridinyl)-2-pyrrolidinone (E29)

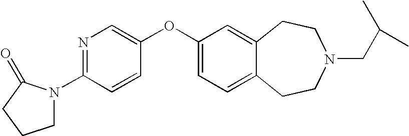

The title product (E29) was prepared from 1-[5-(2,3,4,5-tetrahydro-1H-3-benzazepin-7-yloxy)-2-pyridinyl]-2-pyrrolidinone (0.05 g, 0.155 mmol) (Example 28, Step 3) and 2-methylpropanal (0.022 g, 0.31 mmol) using the method of Example 28, Step 4; MS (ES+) m/e 380 [M+H]⁺.

### EXAMPLE 30

### 1-(5-{[3-(1-Methylethyl)-2,3,4,5-tetrahydro-1H-3-benzazepin-7-yl]oxy}-2-pyridinyl)-2-pyrrolidinone (E30)

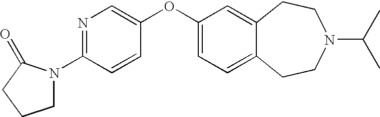

The title product (E30) was prepared from 1-[5-(2,3,4,5-tetrahydro-1H-3-benzazepin-7-yloxy)-2-pyridinyl]-2-pyrrolidinone (0.05 g, 0.155 mmol) (Example 28, Step 3) and acetone (0.018 g, 0.31 mmol) using the method of Example 28, Step 4; MS (ES+) m/e 366 [M+H]⁺.

### EXAMPLE 31

### 1,1-Dimethylethyl 4-({[3-(cyclopropylmethyl)-2,3,4,5-tetrahydro-1H-3-benzazepin-7-yl]oxy}methyl)-1-piperidinecarboxylate (E31)

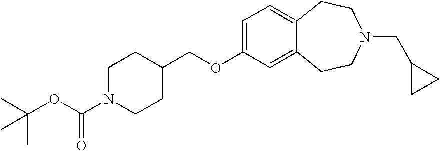

The title product (E31) was prepared as described in Example 5, step 3.

### EXAMPLE 32

### 3-(Cyclopropylmethyl)-7-[(4-iodophenyl)oxy]-2,3,4,5-tetrahydro-1H-3-benzazepine (E32)

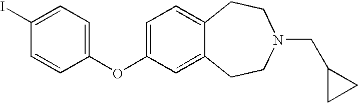

The title product (E32) was prepared as described in Example 18, step 1.

Biological Data

A membrane preparation containing histamine H3 receptors may be prepared in accordance with the following procedures:

(i) Generation of Histamine H3 Cell Line

DNA encoding the human histamine H3 gene (Huvar, A. et al. (1999) Mol. Pharmacol. 55(6), 1101-1107) was cloned into a holding vector, pCDNA3.1 TOPO (InVitrogen) and its cDNA was isolated from this vector by restriction digestion of plasmid DNA with the enzymes BamH1 and Not-1 and ligated into the inducible expression vector pGene (InVitrogen) digested with the same enzymes. The GeneSwitch™ system (a system where in transgene expression is switched off in the absence of an inducer and switched on in the presence of an inducer) was performed as described in U.S. Pat. Nos. 5,364,791; 5,874,534; and 5,935,934. Ligated DNA was transformed into competent DH5α E. coli host bacterial cells and plated onto Luria Broth (LB) agar containing Zeocin™ (an antibiotic which allows the selection of cells expressing the sh ble gene which is present on pGene and pSwitch) at 50 μg ml⁻¹. Colonies containing the re-ligated plasmid were identified by restriction analysis. DNA for transfection into mammalian cells was prepared from 250 ml cultures of the host bacterium containing the pGeneH3 plasmid and isolated using a DNA preparation kit (Qiagen Midi-Prep) as per manufacturers guidelines (Qiagen).

CHO K1 cells previously transfected with the pSwitch regulatory plasmid (InVitrogen) were seeded at 2×10e6 cells per T75 flask in Complete Medium, containing Hams F12 (GIBCOBRL, Life Technologies) medium supplemented with 10% v/v dialysed foetal bovine serum, L-glutamine, and hygromycin (100 μg ml^{31 1}), 24 hours prior to use. Plasmid DNA was transfected into the cells using Lipofectamine plus according to the manufacturers guidelines (InVitrogen). 48 hours post transfection cells were placed into complete medium supplemented with 500 μg ml⁻¹ Zeocin™.

10-14 days post selection 10 nM Mifepristone (InVitrogen), was added to the culture medium to induce the expression of the receptor. 18 hours post induction cells were detached from the flask using ethylenediamine tetra-acetic acid (EDTA; 1:5000; InVitrogen), following several washes with phosphate buffered saline pH 7.4 and resuspended in Sorting Medium containing Minimum Essential Medium (MEM), without phenol red, and supplemented with Earles salts and 3% Foetal Clone II (Hyclone). Approximately 1×10e7 cells were examined for receptor expression by staining with a rabbit polyclonal antibody, 4a, raised against the N-terminal domain of the histamine H3 receptor, incubated on ice for 60 minutes, followed by two washes in sorting medium. Receptor bound antibody was detected by incubation of the cells for 60 minutes on ice with a goat anti rabbit antibody, conjugated with Alexa 488 fluorescence marker (Molecular Probes). Following two further washes with Sorting Medium, cells were filtered through a 50 μm Filcon™ (BD Biosciences) and then analysed on a FACS Vantage SE Flow Cytometer fitted with an Automatic Cell Deposition Unit. Control cells were non-induced cells treated in a similar manner. Positively stained cells were sorted as single cells into 96-well plates, containing Complete Medium containing 500 μg ml⁻¹ Zeocin™ and allowed to expand before reanalysis for receptor expression via antibody and ligand binding studies. One clone, 3H3, was selected for membrane preparation.

(ii) Membrane Preparation From Cultured Cells

All steps of the protocol are carried out at 4° C. and with pre-cooled reagents. The cell pellet is resuspended in 10 volumes of homogenisation buffer (50 mM N-2-hydroxyethylpiperazine-N′-2-ethanesulfonic acid (HEPES), 1 mM ethylenediamine tetra-acetic acid (EDTA), pH 7.4 with KOH, supplemented with 10e-6M leupeptin (acetyl-leucyl-leucyl-arginal; Sigma L2884), 25 μg/ml bacitracin (Sigma B0125), 1 mM phenylmethylsulfonyl fluoride (PMSF) and 2×10e-6M pepstain A (Sigma)). The cells are then homogenised by 2×15 second bursts in a 1 litre glass Waring blender, followed by centrifugation at 500 g for 20 minutes. The supernatant is then spun at 48,000 g for 30 minutes. The pellet is resuspended in homogenisation buffer (4× the volume of the original cell pellet) by vortexing for 5 seconds, followed by homogenisation in a Dounce homogeniser (10-15 strokes). At this point the preparation is aliquoted into polypropylene tubes and stored at −80° C.

A histamine H1 cell line may be generated in accordance with the following procedure:

(iii) Generation of Histamine H1 Cell Line

The human H1 receptor was cloned using known procedures described in the literature [Biochem. Biophys. Res. Commun. 1994, 201(2), 894]. Chinese hamster ovary cells stably expressing the human H1 receptor were generated according to known procedures described in the literature [Br. J. Pharmacol. 1996, 117(6), 1071].

Compounds of the invention may be tested for in vitro biological activity in accordance with the following assays:

(I) Histamine H3 Functional Antagonist Assay (Method A)

For each compound being assayed, in a white walled clear bottom 96 well plate, is added:

(a) 10 μl of test compound (or 10 μl of guanosine 5′-triphosphate (GTP) (Sigma) as non-specific binding control) diluted to required concentration in 10% DMSO;

(b) 60 μl bead/membrane/GDP mix prepared by suspending wheat germ agglutinin-polyvinyltoluene (WGA-PVT) scintillation proximity assay (SPA) beads at 100 mg/ml in assay buffer (20 mM N-2-Hydroxyethylpiperazine-N′-2-ethanesulfonic acid (HEPES)+100 mM NaCl+10 mM MgCl₂, pH 7.4 NaOH) followed by mixing with membrane (prepared in accordance with the methodology described above) and diluting in assay buffer to give a final volume of 60 μl which contains 10 μg protein and 0.5 mg bead per well—mixture is pre-mixed at 4° C. for 30 minutes on a roller and just prior to addition to the plate, 10 μM final concentration of guanosine 5′ diphosphate (GDP) (Sigma; diluted in assay buffer) is added; The plate is incubated at room temperature to equilibrate antagonist with receptor/beads by shaking for 30 minutes followed by addition of:

(c) 10 μl histamine (Tocris) at a final concentration of 0.3 μM; and

(d) 20 μl guanosine 5′ [y35-S] thiotriphosphate, triethylamine salt (Amersham; radioactivity concentration=37 kBq/μl or 1 mCi/ml; Specific Activity 1160 Ci/mmol) diluted to 1.9 nM in assay buffer to give 0.38 nM final.

The plate is then incubated on a shaker at room temperature for 30 minutes followed by centrifugation for 5 minutes at 1500 rpm. The plate is read between 3 and 6 hours after completion of centrifuge run in a Wallac Microbeta counter on a 1 minute normalised tritium count protocol. Data is analysed using a 4-parameter logistic equation. Basal activity used as minimum i.e. histamine not added to well.

(II) Histamine H3 Functional Antagonist Assay (Method B)

For each compound being assayed, in a solid white 384 well plate, is added:

(a) 5 μl of test compound diluted to the required concentration in 10% DMSO (or 5 μl 10% DMSO as a control); and

(b) 30 μl bead/membrane/GDP mix prepared by mixing Wheat Germ Agglutinin Polystyrene LeadSeeker® (WGA PS LS) scintillation proximity assay (SPA) beads with membrane (prepared in accordance with the methodology described above) and diluting in assay buffer (20 mM N-2-Hydroxyethylpiperazine-N′-2-ethanesulfonic acid (HEPES)+100 mM NaCl+10 mM MgCl₂, pH 7.4 NaOH) to give a final volume of 30 μl which contains 5 μg protein and 0.25 mg bead per well, incubating at 4° C. for 30 minutes on a roller and, just prior to addition to the plate, adding 10 μM final concentration of guanosine 5′ diphosphate (GDP) (Sigma; diluted in assay buffer).

The plates were then incubated at room temperature for 30 minutes on a shaker followed by addition of:

(c) 15 μl 0.38nM [³⁵S]-GTPγS (Amersham; Radioactivity concentration=37 MBq/ml; Specific activity=1160 Ci/mmol), histamine (at a concentration that results in the final assay concentration of histamine being EC₈₀).

After 2-6 hours, the plate is centrifuged for 5 min at 1500 rpm and counted on a Viewlux counter using a 613/55 filter for 5 min/plate. Data is analysed using a 4-parameter logistical equation. Basal activity used as minimum i.e. histamine not added to well.

(III) Histamine H3 Functional Antagonist Assay (Method C)

For each compound being assayed, in a solid white 384 well plate, is added:

(a) 0.5 μl of test compound diluted to the required concentration in DMSO (or 0.5 μl DMSO as a control);

(b) 30 μl bead/membrane/GDP mix prepared by mixing Wheat Germ Agglutinin Polystyrene LeadSeeker® (WGA PS LS) scintillation proximity assay (SPA) beads with membrane (prepared in accordance with the methodology described above) and diluting in assay buffer (20 mM N-2-Hydroxyethylpiperazine-N′-2-ethanesulfonic acid (HEPES)+100 mM NaCl+10 mM MgCl₂, pH 7.4 NaOH) to give a final volume of 30 μl which contains 5 μg protein and 0.25 mg bead per well, incubating at room temperature for 60 minutes on a roller and, just prior to addition to the plate, adding 10 μM final concentration of guanosine 5′ diphosphate (GDP) (Sigma; diluted in assay buffer);

(c) 15 μl 0.38nM [³⁵S]-GTPγS (Amersham; Radioactivity concentration=37MBq/ml; Specific activity=1160 Ci/mmol), histamine (at a concentration that results in the final assay concentration of histamine being EC₈₀).

After 2-6 hours, the plate is centrifuged for 5 min at 1500 rpm and counted on a Viewlux counter using a 613/55 filter for 5 min/plate. Data is analysed using a 4-parameter logistical equation. Basal activity used as minimum i.e. histamine not added to well.

(IV) Histamine H1 Functional Antagonist Assay (Method A)

The compound is assayed in a black walled clear bottom 384-well plate with cells seeded at 10000 cells/well. Tyrodes buffer is used throughout (NaCl 145 mM, KCl 2.5 mM, HEPES 10 mM, glucose 10 mM, MgCl₂ 1.2 mM, CaCl₂ 1.5 mM, probenecid 2.5 mM, pH adjusted to 7.40 with NaOH 1.0 M). Each well is treated with 10 μl of a solution of FLUO4AM (10 μM in Tyrodes buffer at pH 7.40) and plates are then incubated for 60 minutes at 37° C. Wells are then washed with Tyrodes buffer using a EMBLA cell washer system, leaving 40 μl buffer in each well, and then treated with 10 μl of test compound in Tyrodes buffer. Each plate is incubated for 30 min to allow equilibration of the test compound with the receptor. Each well is then treated with 10 μl of histamine solution in Tyrodes buffer.

Functional antagonism is indicated by a suppression of histamine induced increase in fluorescence, as measured by the FLIPR system (Molecular Devices). By means of concentration effect curves, functional potencies are determined using standard pharmacological mathematical analysis.

(V) Histamine H1 Functional Antagonist Assay (Method B)

The histamine H1 cell line was seeded into non-coated black-walled clear bottom 384-well tissue culture plates in alpha minimum essential medium (Gibco/Invitrogen, cat no. 22561-021), supplemented with 10% dialysed foetal calf serum (Gibco/lnvitrogen cat no. 12480-021) and 2 mM L-glutamine (Gibco/Invitrogen cat no 25030-024) and maintained overnight at 5% CO₂, 37° C.

Excess medium was removed from each well to leave 10 μl. 30 μl loading dye (250 μM Brilliant Black, 2 μM Fluo-4 diluted in Tyrodes buffer+probenecid (145 mM NaCl, 2.5 mM KCl, 10 mM HEPES, 10 mM D-glucose, 1.2 mM MgCl₂, 1.5 mM CaCl₂, 2.5 mM probenecid, pH adjusted to 7.40 with NaOH 1.0 M)) was added to each well and the plates were incubated for 60 minutes at 5% CO₂, 37° C.

10 μl of test compound, diluted to the required concentration in Tyrodes buffer+probenecid (or 10 μl Tyrodes buffer+probenecid as a control) was added to each well and the plate incubated for 30 min at 37° C., 5% CO₂. The plates were then placed into a FLIPR™ (Molecular Devices, UK) to monitor cell fluorescence (λₑₓ=488 nm, λ_{EM}=540 nm) in the manner described in Sullivan et al. (In: Lambert DG (ed.), Calcium Signaling Protocols, New Jersey: Humana Press, 1999, 125-136) before and after the addition of 10 μl histamine at a concentration that results in the final assay concentration of histamine being EC₈₀.

Functional antagonism is indicated by a suppression of histamine induced increase in fluorescence, as measured by the FLIPR™ system (Molecular Devices). By means of concentration effect curves, functional affinities are determined using standard pharmacological mathematical analysis.

Results

The compounds of Examples E19 and E20 were tested in the histamine H3 functional antagonist assay (method A) and exhibited antagonism>⁷ pK_{b}. More particularly, the compound of Example 19 exhibited antagonism>8 pK_{b}.

The compounds of Examples E1, E2, E5, E10-14, E16-18, E21, E23-26 and E28-30 were tested in the histamine H3 functional antagonist assay (method B) and exhibited antagonism>7 pK_{b}. More particularly, the compounds of Examples E1, E5, E10-14. E16-18, E23-24 and E28-30 exhibited antagonism>8 pK_{b}. Most particularly, the compounds of Examples E1, E13 and E14 exhibited antagonism>9 pK_{b}.

The compounds of Examples E1, E3-4, E6-9, E11-18, E22-24 and E27-29 were tested in the histamine H3 functional antagonist assay (method C) and exhibited antagonism>7 pK_{b}. More particularly, the compounds of Examples E1 and E15 exhibited antagonism>9 pK_{b}.

The compounds of Example E19 was tested in the histamine H1 functional antagonist assay (method A) and exhibited antagonism<6.5 pK_{b}.

The compounds of Examples E1-18, E22-24 and E26-30 were tested in the histamine H1 functional antagonist assay (method B) and exhibited antagonism<6.5 pK_{b}.

What is claimed is:

1.-9. (canceled)

10. A compound of formula (I) or a pharmaceutically acceptable salt thereof: 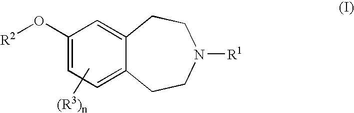
wherein:
R¹ represents —C₂₋₇ alkyl or —(CH₂)ₘ—C₃₋₇ cycloalkyl;
R² represents —X—C₃₋₈ cycloalkyl, —X-aryl, —X-heteroaryl, —X-heterocyclyl, —X—C₃₋₈ cycloalkyl-Y—C₃₋₈ cycloalkyl, —X—C₃₋₈ cycloalkyl-Y-aryl, —X—C₃₋₈ cycloalkyl-Y-heteroaryl, —X—C₃₋₈ cycloalkyl-Y-heterocyclyl, —X-aryl-Y—C₃₋₈ cycloalkyl, —X-aryl-Y-aryl, —X-aryl-Y-heteroaryl, —X-aryl-Y-heterocyclyl, —X-heteroaryl-Y—C₃₋₈ cycloalkyl, —X-heteroaryl-Y-aryl, —X-heteroaryl-Y-heteroaryl, —X-heteroaryl-Y-heterocyclyl, —X-heterocyclyl-Z-aryl, —X-heterocyclyl-Y—C₃₋₈ cycloalkyl, —X-heterocyclyl-Y-heteroaryl or —X-heterocyclyl-W-heterocyclyl, such that R² is linked to O via a carbon atom;
W represents a bond, C₁₋₆ alkyl, CO, COC₂₋₆ alkenyl, O or SO₂;
X represents a bond or methyl;
Y represents a bond, C₁₋₆ alkyl, CO, COC₂₋₆ alkenyl, O or SO₂;
Z represents a bond, CO, COC₂₋₆ alkenyl, O or SO₂;
R³ represents halogen, C₁₋₆ alkyl, C₁₋₆ alkoxy, cyano, amino or trifluoromethyl;
m represents an integer from 1-3;
n is 0, 1 or 2;
wherein said alkyl groups of R¹ may be optionally substituted by one or more substituents which may be the same or different and which are selected from the group consisting of halogen, cyano, C₁₋₆ alkyl, C₁₋₆ alkoxy, haloC₁₋₆ alkyl and haloC₁₋₆ alkoxy;
wherein said cycloalkyl, aryl, heteroaryl and heterocyclyl groups of R² may be optionally substituted by one or more substituents which may be the same or different, and which are selected from the group consisting of halogen, hydroxy, cyano, nitro, ═O, trifluoromethyl, trifluoromethoxy, fluoromethoxy, difluoromethoxy, C₁₋₆ alkyl, pentafluoroethyl, C₁₋₆ alkoxy, arylC₁₋₆ alkoxy, C₁₋₆ alkylthio, C₁₋₆ alkoxyC₁₋₆ alkyl, C₃₋₇ cycloalkylC₁₋₆ alkoxy, C₁₋₆ alkanoyl, C₁₋₆ alkoxycarbonyl, C₁₋₆ alkylsulfonyl, C₁₋₆ alkylsulfinyl, C₁₋₆ alkylsulfonyloxy, C₁₋₆ alkylsulfonylC₁₋₆ alkyl, sulfonyl, arylsulfonyl, arylsulfonyloxy, arylsulfonylC₁₋₆ alkyl, aryloxy, C₁₋₆ alkylsulfonamido, C₁₋₆ alkylamino, C₁₋₆ alkylamido, —R⁴, —CO₂R⁴, —COR⁴, C₁₋₆ alkylsulfonamidoC₁₋₆ alkyl, C₁₋₆ alkylamidoC₁₋₆ alkyl, arylsulfonamido, arylcarboxamido, arylsulfonamidoC₁₋₆ alkyl, arylcarboxamidoC₁₋₆ alkyl, aroyl, aroylC₁₋₆ alkyl, arylC₁₋₆ alkanoyl, or a group —NR⁵R⁶, —C₁₋₆ alkyl-NR⁵R⁶, —C₃₋₈ cycloalkyl-NR⁵R⁶, —CONR⁵R⁶, —NR⁵COR⁶, —NR⁵SO₂R⁶, —OCONR⁵R⁶, —NR⁵CO₂R⁶, —NR⁴CONR⁵R⁶ and —SO₂NR⁵R⁶, wherein R⁴, R⁵ and R⁶ independently represent hydrogen, C₁₋₆ alkyl, —C₃₋₈ cycloalkyl, —C₁₋₆ alkyl-C₃₋₈ cycloalkyl, aryl, heterocyclyl or heteroaryl or wherein —NR⁵R⁶ may represent a nitrogen containing heterocyclyl group, wherein said R⁴, R⁵ and R⁶ groups may be optionally substituted by one or more substituents which may be the same or different, and which are selected from the group consisting of halogen, hydroxy, C₁₋₆ alkyl, C₁₋₆ alkoxy, cyano, amino, ═O and trifluoromethyl;
or solvates thereof.

11. A compound as defined in claim 10 which is:
1-(5-{[3-(cyclopropylmethyl)-2,3,4,5-tetrahydro-1H-3-benzazepin-7-yl]oxy}-2-pyrazinyl)-2-pyrrolidinone;
3-(1-methylethyl)-7-[(phenylmethyl)oxy]-2,3,4,5-tetrahydro-1H-3-benzazepine;
3-(2-methylpropyl)-7-[(phenylmethyl)oxy]-2,3,4,5-tetrahydro-1H-3-benzazepine;
3-Ethyl-7-[(phenylmethyl)oxy]-2,3,4,5-tetrahydro-1H-3-benzazepine;
3-(cyclopropylmethyl)-7-[(4-piperidinylmethyl)oxy]-2,3,4,5-tetrahydro-1H-3-benzazepine;
4-{[4-({[3-(cyclopropylmethyl)-2,3,4,5-tetrahydro-1H-3-benzazepin-7-yl]oxy}methyl)-1-piperidinyl]carbonyl}benzonitrile;
3-(cyclopropylmethyl)-7-[({1-[(4-fluorophenyl)carbonyl]-4-piperidinyl}methyl)oxy]-2,3,4,5-tetrahydro-1H-3-benzazepine;
7-({[1-(cyclopropylcarbonyl)-4-piperidinyl]methyl}oxy)-3-(cyclopropylmethyl)-2,3,4,5-tetrahydro-1H-3-benzazepine;
3-(cyclopropylmethyl)-7-({[1-(tetrahydro-2H-pyran-4-ylcarbonyl)-4-piperidinyl]methyl}oxy)-2,3,4,5-tetrahydro-1H-3-benzazepine;
1-(6-{[3-(1-methylethyl)-2,3,4,5-tetrahydro-1H-3-benzazepin-7-yl]oxy}-3-pyridinyl)-2-pyrrolidinone;
1-(6-{[3-(2-methylpropyl)-2,3,4,5-tetrahydro-1H-3-benzazepin-7-yl]oxy}-3-pyridinyl)-2-pyrrolidinone;
1-(6-{[3-(2,2-dimethylpropyl)-2,3,4,5-tetrahydro-1H-3-benzazepin-7-yl]oxy}-3-pyridinyl)-2-pyrrolidinone;
1-(6-{[3-(cyclopropylmethyl)-2,3,4,5-tetrahydro-1H-3-benzazepin-7-yl]oxy}-3-pyridinyl)-2-pyrrolidinone;
1-{6-[(3-ethyl-2,3,4,5-tetrahydro-1H-3-benzazepin-7-yl)oxy]-3-pyridinyl}-2-pyrrolidinone;
1-(6-{[3-(1-methylpropyl)-2,3,4,5-tetrahydro-1H-3-benzazepin-7-yl]oxy}-3-pyridinyl)-2-pyrrolidinone;
1-(6-{[3-(cyclobutylmethyl)-2,3,4,5-tetrahydro-1H-3-benzazepin-7-yl]oxy}-3-pyridinyl)-2-pyrrolidinone;
3-(cyclopropylmethyl)-7-{[5-(3-methyl-1,2,4-oxadiazol-5-yl)-2-pyridinyl]oxy}-2,3,4,5-tetrahydro-1H-3-benzazepine;
1-(4-{[3-(cyclopropylmethyl)-2,3,4,5-tetrahydro-1H-3-benzazepin-7-yl]oxy}phenyl)-3-methyl-2-imidazolidinone;
3-(cyclopropylmethyl)-7-[(phenylmethyl)oxy]-2,3,4,5-tetrahydro-1H-3-benzazepine;
7-[(3-cyclohexylpropyl)oxy]-3-(cyclopropylmethyl)-2,3,4,5-tetrahydro-1H-3-benzazepine;
3-(cyclopropylmethyl)-7-(phenyloxy)-2,3,4,5-tetrahydro-1H-3-benzazepine;
Ethyl 4-{[3-(cyclopropylmethyl)-2,3,4,5-tetrahydro-1H-3-benzazepin-7-yl]oxy}benzoate;
6-{[3-(cyclopropylmethyl)-2,3,4,5-tetrahydro-1H-3-benzazepin-7-yl]oxy}-N-methyl-3-pyridinecarboxamide;
5-{[3-(cyclopropylmethyl)-2,3,4,5-tetrahydro-1H-3-benzazepin-7-yl]oxy}-N-methyl-2-pyrazinecarboxamide;
1,1-dimethylethyl 4-{[3-(cyclopropylmethyl)-2,3,4,5-tetrahydro-1H-3-benzazepin-7-yl]oxy}-1-piperidinecarboxylate;
3-(cyclopropylmethyl)-7-(4-piperidinyloxy)-2,3,4,5-tetrahydro-1H-3-benzazepine;
4-[(4-{[3-(cyclopropylmethyl)-2,3,4,5-tetrahydro-1H-3-benzazepin-7-yl]oxy}-1-piperidinyl)carbonyl]benzonitrile;
1-(5-{[3-(cyclopropylmethyl)-2,3,4,5-tetrahydro-1H-3-benzazepin-7-yl]oxy}-2-pyridinyl)-2-pyrrolidinone;
1-(5-{[3-(2-methylpropyl)-2,3,4,5-tetrahydro-1H-3-benzazepin-7-yl]oxy}-2-pyridinyl)-2-pyrrolidinone;
1-(5-{[3-(1-methylethyl)-2,3,4,5-tetrahydro-1H-3-benzazepin-7-yl]oxy}-2-pyridinyl)-2-pyrrolidinone;
1,1-dimethylethyl 4-({[3-(cyclopropylmethyl)-2,3,4,5-tetrahydro-1H-3-benzazepin-7-yl]oxy}methyl)-1-piperidinecarboxylate;
3-(cyclopropylmethyl)-7-[(4-iodophenyl)oxy]-2,3,4,5-tetrahydro-1H-3-benzazepine;
or a pharmaceutically acceptable salt or solvate thereof.

12. A pharmaceutical composition which comprises the compound of claim 10 or a pharmaceutically acceptable salt or solvate thereof and a pharmaceutically acceptable carrier or excipient.

13. A method of treatment of neurological diseases which comprises administering to a host in need thereof an effective amount of a compound claim 10 or a pharmaceutically acceptable salt or solvate thereof.

14. A process for the preparation of a compound of formula (I) or a pharmaceutically acceptable salt or solvate thereof, which process comprises:
(a) reacting a compound of formula (II) 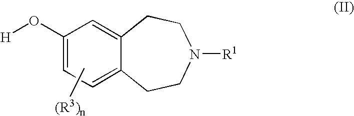
wherein R¹, R³ and n are as defined in claim 10, with a compound of formula R^{2′}-L¹, wherein R^{2′} is as defined in claim 10 for R² or a group convertible thereto and L¹ represents a suitable leaving group such as a halogen atom or an optionally activated hydroxyl group;
(b) reacting a compound of formula (III) 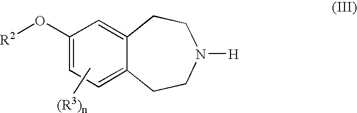
wherein R², R³ and n are as defined in claim 10, with a compound of formula R^{1′}-L², wherein R¹ is as defined in claim 10 for R¹ or a group convertible thereto and L² represents a suitable leaving group such as a halogen atom; or
(c) reacting a compound of formula (III) as defined above, with a ketone of formula R^{1″}═O, wherein R^{1″} is ═C₂₋₇ alkyl or ═(CH₂)ₘ—C₃₋₇ cycloalkyl or a group convertible thereto; or
(d) deprotecting a compound of formula (I) which is protected; or
(e) interconversion from one compound of formula (I) to another.
